(19) United States
(12) Patent Application Publication
(10) Pub. No.: US 2025/0213808 A1
(43) Pub. Date: Jul. 3, 2025

(54) RESPIRATORY SUPPORT APPARATUS

(71) Applicant: EDWARD D LIN, OSPREY, FL (US)

(72) Inventor: EDWARD D LIN, OSPREY, FL (US)

(21) Appl. No.: 18/398,071

(22) Filed: Dec. 27, 2023

(51) Int. Cl. A61M 16/00; A61M 16/06; A61M 16/20

(52) U.S. Cl. CPC A61M 16/009; A61M 16/0078; A61M 16/06; A61M 16/208; A61M 2202/0208

(57) ABSTRACT

Respiratory support apparatus and related methods of use are disclosed herein that include a manifold and a facemask. The manifold is attachable to the facemask and includes valves that control the inflow and outflow of gas through the manifold and facemask. The valves are positioned to minimize the volume of the manifold to allow flushing of the manifold during a portion of exhalation. A volume of the facemask is also minimized to allow flushing of the facemask during the portion of exhalation. The flow rate of the respiratory gas and a dead space volume comprising the manifold volume of the manifold and a facemask volume of the facemask is configured to equate generally a detention time and a flushing time.

### BACKGROUND OF THE INVENTION

### Field

This disclosure relates to apparatus and related methods for supplying breathable gas to a user, and more specifically, to apparatus and related methods for supporting respiration in users suffering from respiratory deficiencies.

### Background

Hypoxia, a deficiency in the amount of oxygen reaching body tissues, is a common serious medical condition that may have many causes. Acute hypoxia may threaten life, and chronic hypoxia may severely impair quality of life while causing steady systemic health deterioration. The air hunger of hypoxia is a most uncomfortable, dreaded sensation. Patients having a variety of acute or chronic conditions may develop significant hypoxia that requires respiratory support as treatment.

High flow nasal cannula (HFNC) has become recognized as a relatively gentler, better option than ventilators for treatment of hypoxia as a result of the extensive adverse experience of COVID patients on ventilators. The HFNC relies on spontaneous breathing and requires a respiratory gas flow rate of about 60 L/min., or 86,400 L/day, which limits the user of HFNC to critical care settings or other institutional settings. There was an oxygen shortage crisis in the US in 2021-22 due to the high consumption of oxygen at least in part due to use of HFNC in treating COVID patients. In addition, HFNC presents a substantial contamination risk due to washout from the pharynx, so that HFNC should be used in a room with negative ventilation to evacuate the washout. However, the availability of such facilities is very limited thus further limiting the use of HFNC.

Oxygen masks are also used ubiquitously in treating hypoxia. However, oxygen masks may not be very effective in delivering oxygen (O₂) for a number of reasons, such that oxygen masks may often fail to provide adequate treatment of hypoxia.

Thus, there is a need for a more efficacious oxygenation devices that can be deployed [1] early in the hypoxic pathophysiologic process, [2] at home before hospital admission becomes necessary, and [3] does not require specialized equipment or skilled healthcare personnel. This inventor, a board-certified anesthesiologist, set to decipher why, after more than eight decades, there is still a lack of effective high-oxygenation devices outside of the ICU. There still remains a need for improved apparatus and related methods for respiratory support that achieve a clinically supportive oxygen saturation in a spontaneously breathing hypoxic patient.

### BRIEF SUMMARY OF THE INVENTION

These and other needs and disadvantages may be overcome by the methods and related apparatus disclosed herein. Additional improvements and advantages may be recognized by those of ordinary skill in the art upon study of the present disclosure.

The respiratory support apparatus disclosed herein includes a manifold defining a manifold chamber and a facemask in mechanical cooperation with the manifold, in various aspects. The facemask defines a facemask chamber in fluid communication with the manifold chamber, and the facemask is adapted to enclose an inspiratory aperture of a user in order to communicate a respiratory gas into alveolar regions of the user, in various aspects. The respiratory gas comprises oxygen at a concentration greater than that of ambient air, and the user is spontaneously breathing, in various aspects. The respiratory support apparatus includes a bag defining a bag reservoir in fluid communication with the respiratory gas, in various aspects. In various aspects, a check valve cooperates with the manifold. The check valve is configured to allow communication of the respiratory gas into the manifold chamber during inhalation, and the check valve is configured to block communication of the respiratory gas into the manifold chamber to fill the bag reservoir with respiratory gas during a portion of exhalation wherein the bag reservoir is less than completely filled with respiratory gas, in various aspects. The check valve is configured to allow communication of the respiratory gas into the manifold chamber during another portion of exhalation wherein the bag reservoir is completely filled with the respiratory gas, in various aspects. A second check valve cooperates with the manifold and is configured to allow communication of outflow gas from the manifold chamber to the ambient environment during exhalation and block communication of ambient air from the ambient environment into the manifold chamber during inhalation, in various aspects. A third check valve cooperates with the manifold to act as an anti-asphyxiation valve, and the third check valve may open to deliver ambient air into the manifold chamber during inhalation as triggered by exhaustion of respiratory gas within the bag reservoir, in various aspects.

The respiratory support apparatus includes a first arm defining a first arm passage, a second arm defining a second arm passage, a third arm defining a third arm passage, and a fourth arm defining a fourth arm passage, the first arm, the second arm, and the third arm being coplanar and disposed radially with and perpendicular to the fourth arm, the first arm and the third arm being perpendicular to the second arm, the fourth arm being attachable to a facemask conduit of a facemask to fluidly communicate between the manifold chamber and a facemask chamber of the facemask, in various aspects. The manifold chamber of the manifold is bounded by a downstream side of the first check valve received within the first arm passage of the first arm, an upstream side of the second check valve received in the second arm passage of the second arm, and a downstream side of the third check valve received within the third arm passage of the third arm, in various aspects. The downstream side of the first check valve. the upstream side of the second check valve, and the downstream side of the third check valve are positioned generally at a circumference of the fourth arm passage in order to minimize a manifold volume of the manifold chamber, in various aspects. Related methods of use of the respiratory support apparatus are also disclosed herein.

This summary is presented to provide a basic understanding of some aspects of the apparatus and methods disclosed herein as a prelude to the detailed description that follows below. Accordingly, this summary is not intended to identify key elements of the apparatus and methods disclosed herein or to delineate the scope thereof.

### BRIEF DESCRIPTION OF THE DRAWINGS

FIG. 1 illustrates by Cartesian plot and semi-schematically certain exemplary anatomical features of a human respiratory system along with an exemplary respiratory cycle;

FIG. 2A illustrates by perspective view an exemplary implementation of a respiratory support apparatus;

FIG. 2B illustrates by another perspective view the exemplary respiratory support apparatus of FIG. 2A;

FIG. 2C illustrates by perspective view portions of the exemplary respiratory support apparatus of FIG. 2A;

FIG. 2D illustrates by cut-away perspective view portions of the exemplary respiratory support apparatus of FIG. 2A;

FIG. 3A illustrates by cut-away top view a manifold that comprises a portion of the exemplary respiratory support apparatus of FIG. 2A;

FIG. 3B illustrates by cut-away side view through cutline 3B-3B of FIG. 3A a portion of the exemplary respiratory support apparatus of FIG. 2A;

FIG. 4 illustrates by plan view a facemask comprising a portion of the exemplary respiratory support apparatus of FIG. 2A;

FIG. 5A illustrates the facemask of FIG. 4 by cut-away view through cutline 5A-5A of FIG. 4;

FIG. 5B illustrates the facemask of FIG. 4 by cut-away view through cutline 5B-5B of FIG. 4;

FIG. 6A illustrates by cut-away perspective view a check valve comprising a portion of the exemplary respiratory support apparatus of FIG. 2A, the check valve being illustrated in a closed position;

FIG. 6B illustrates by cut-away perspective view the check valve of FIG. 6A in an open position;

FIG. 7A illustrates by cut-away perspective view portions of another exemplary implementation of a respiratory support apparatus;

FIG. 7B illustrates by perspective view other portions of the exemplary respiratory support apparatus of FIG. 7A;

FIG. 7C illustrates by exploded perspective view portions of the exemplary respiratory support apparatus of FIG. 7A;

FIG. 7D illustrates by cut-away plan view portions of the exemplary respiratory support apparatus of FIG. 7A;

FIG. 8A illustrates by schematic diagram the exemplary respiratory support apparatus of FIG. 2A in a first operational state;

FIG. 8B illustrates by schematic diagram the exemplary respiratory support apparatus of FIG. 2A in a second operational state;

FIG. 8C illustrates by schematic diagram the exemplary respiratory support apparatus of FIG. 2A in a third operational state;

FIG. 8D illustrates by schematic diagram the exemplary respiratory support apparatus of FIG. 2A in a fourth operational state;

FIG. 9 illustrates by process flow chart exemplary methods of operation of the exemplary respiratory support apparatus of FIGS. 2A, 7A; and,

FIG. 10 illustrates by process flow chart other exemplary methods of operation of the exemplary respiratory support apparatus of FIGS. 2A, 7A.

The Figures are exemplary only, and the implementations illustrated therein are selected to facilitate explanation. The number, position, relationship and dimensions of the elements shown in the Figures to form the various implementations described herein, as well as dimensions and dimensional proportions to conform to specific force, weight, strength, flow and similar requirements are explained herein or are understandable to a person of ordinary skill in the art upon study of this disclosure. Where used in the various Figures, the same numerals designate the same or similar elements. Furthermore, when the terms “top,” “bottom,” “right,” “left,” “forward,” “rear,” “first,” “second,” “inside,” “outside,” and similar terms are used, the terms should be understood in reference to the orientation of the implementations shown in the drawings and are utilized to facilitate description thereof. Use herein of relative terms such as generally, about, approximately, essentially, may be indicative of engineering, manufacturing, or scientific tolerances such as ±0.1%, ±1%, ±2.5%, ±5%, or other such tolerances, as would be recognized by those of ordinary skill in the art upon study of this disclosure.

### DETAILED DESCRIPTION OF THE INVENTION

A respiratory support apparatus that includes a manifold attachable onto a facemask for communication of fluid between a manifold chamber defined by the manifold and a facemask chamber defined by the facemask is disclosed herein. In various aspects, a dead space volume comprising a manifold volume of the manifold and a facemask volume of the facemask is minimized in order to reduce potential additive effects of the dead space volume onto the anatomical dead space that affect oxygen delivery.

The respiratory support apparatus and related methods of use, in various aspects, are configured to maximize delivery of oxygen to alveolar regions of the lungs under various operating conditions. In addition, the respiratory support apparatus allows measurement of attributes of exhalant such as the concentration of carbon dioxide (EtCO2), ketones and alcohol, in various aspects.

Respiratory gas is communicated into the manifold chamber of the manifold from a gas source, and check valves cooperate with the manifold chamber to control the flow of respiratory gas through the manifold into the facemask chamber and the flow of outflow gas from the facemask chamber through the manifold and into the ambient environment as a user breathes, in various aspects. One of the check valves cooperates with the manifold chamber to flow ambient air into the manifold chamber when insufficient respiratory gas is available for the user to inhale thereby functioning as an anti-asphyxiation valve, in various aspects. In various aspects, the check valve functioning as the anti-asphyxiation valve is configured to assume an open position only during a terminal portion of inhalation, thereby communicating the respiratory gas into an alveolar region of the user and the ambient air only into an anatomical dead space of the user. Opening of the anti-asphyxiation valve during the terminal portion of inhalation avoids dilution of respiratory gas that is communicated into the alveolar region with ambient air while communicating the ambient air into an anatomical dead space of the user. In various aspects, the valve member may be configured to position the anti-asphyxiation valve from a closed position into an open position to communicate the respiratory gas into an alveolar region of the user and the ambient air only into an anatomical dead space of the user by selecting the Shore hardness of the valve member or otherwise configuring the valve member. In certain aspects, the bag reservoir of a bag in communication with the supply of respiratory gas may be sized to position the anti-asphyxiation valve from a closed position into an open position to communicate the respiratory gas into an alveolar region of the user and the ambient air only into an anatomical dead space of the user.

In various aspects, multiple check valves open to flow respiratory gas through the manifold and through the facemask flushing outflow gas from the manifold chamber and the facemask chamber during exhalation. This flushing may allow increased inhalation of respiratory gas into the alveolar regions where oxygen exchange takes place.

In some aspects, the facemask may be configured to have a concave contour to its “cone” or “dome” to minimize the facemask volume thereof in order to enhance flushing of outflow gas from the facemask chamber. The facemask may be configured to have a concave contour configured to approximate a facial contour of a face.

In other aspects, the facemask may be, for example, a standard anesthesia facemask, a resuscitation facemask, or other leak resistant facemask with or without an inflatable cushion, and the manifold may be configured to connect to a facemask conduit of the facemask. The manifold chamber may be configured to reduce the volume thereof in order to enhance flushing of outflow gas from the manifold chamber.

The respiratory support apparatus disclosed herein may be used for oxygen supplementation of spontaneously breathing users, in various aspects. In such uses, the respiratory support apparatus may provide a higher fraction (up to 100%) of inspired oxygen (FiO2) than nasal cannula (about 35%) while being non-invasive. Because, in various aspects, the respiratory support apparatus is non-invasive and relies on spontaneous respiration of the user, the respiratory support apparatus disclosed herein may provide advantages over ventilator-mediated respiration, including: [1] elimination of risk of respiratory arrest if endotracheal tube is dislodged while the user remains paralyzed and/or sedated [2] elimination of ventilator-dependency and of inability to be weaned off of mechanical ventilation, [3] no circumvention of natural air filtering and immune defenses provided by nasal turbinates, lymphoid tissue, and pharyngeal mucosa as would occur with use of an endotracheal tube, the endotracheal tube being associated with high risk of nosocomial infections; and [4] reduction of cost associated with ventilator use and ICU stay. Because the respiratory support apparatus may be single use, in various aspects, disposal following use may aid infection control.

As used herein, a user is defined as a person to whom the respiratory support apparatus is attached or is adapted for attachment. In certain aspects, a healthcare provider may employ the respiratory support apparatus in treating the user, or the healthcare provider may be the user, for example, for protection against infection transmission from others. Healthcare provider may be, for example, a physician, physician's assistant, nurse, or respiratory therapist.

As used herein, the terms distal and proximal are defined from the point of view of the healthcare provider treating the user with the respiratory support apparatus. A distal portion of the respiratory support apparatus is oriented toward the user (e.g., the person being treated) while a proximal portion of the respiratory support apparatus is oriented toward the healthcare provider. In general, a distal portion of a structure is closest to the user (e.g., the patient) while a proximal portion of the structure is closest to the healthcare provider treating the user.

Ambient pressure p_{amb}, as used herein, refers to the pressure in a region surrounding the respiratory support apparatus. Ambient pressure p_{amb}, for example, may refer to atmospheric pressure, hull pressure within an aircraft where the respiratory support apparatus is being utilized, or pressure maintained within a building or other structure where the respiratory support apparatus is being utilized. Ambient pressure p_{amb} may vary, for example, with elevation or weather conditions. Unless specifically stated, pressure as used herein refers to gauge pressure, that is, pressure relative to ambient pressure p_{amb}. Positive pressures indicate pressures greater than ambient pressure p_{amb}, and negative pressures indicate pressures less than ambient pressure p_{amb}. Respiratory gas, as used herein, includes oxygen at a concentration greater than that of atmospheric air, which is approximately 21%. Respiratory gas, oxygen, and similar terms may be used interchangeably herein.

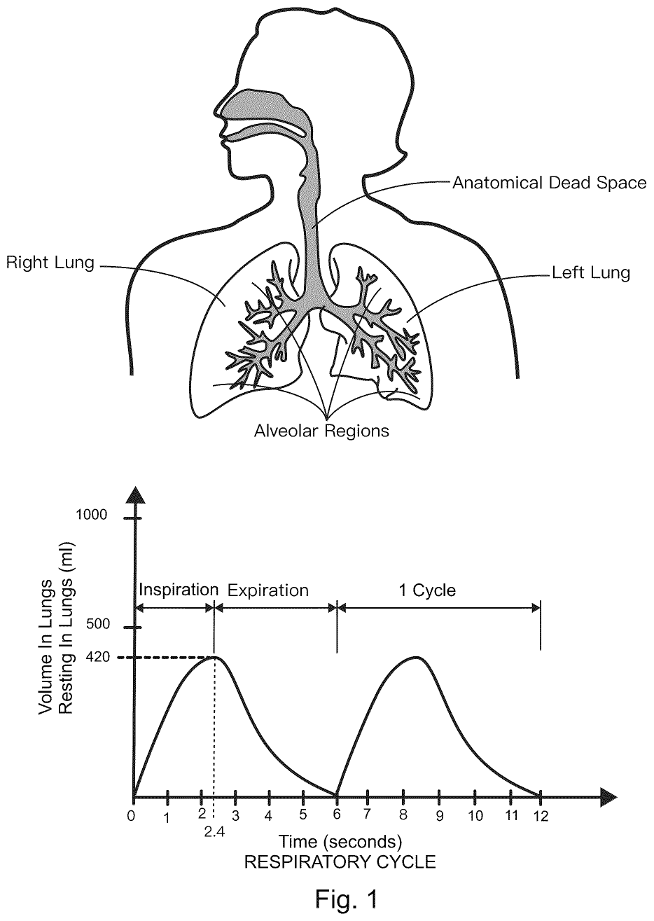

In order to understand hypoxia and its treatment, it is important to appreciate a few facts about the physiology of the human respiratory system. As illustrated in FIG. 1, the anatomy of the human respiratory system may be divided into two parts. One part of the human respiratory system includes the alveolar region of each lung wherein the alveolar region comprises the peripheral microscopic grape-like clusters of distensible air sacs. Oxygen exchange occurs in the alveolar region of the lungs. The other part of the human respiratory system includes the anatomical dead space (ADS). The ADS is illustrated in gray in FIG. 1. The ADS includes, for example, the oropharyngeal and nasopharyngeal spaces, trachea, bronchi and bronchioles generally through which air is being communicated with the alveolar regions during breathing. It is important to appreciate that virtually no oxygen exchange occurs in the ADS.

### Example 1

Example 1 provides a baseline example of human respiration as illustrated by the exemplary respiratory cycle depicted in FIG. 1. The respiratory cycle of FIG. 1 is exemplary of a resting 70 kg adult male. The volume of each inspiration is termed tidal volume (TV) and may be generally in a range of 6 ml/kg-7 ml/kg. As illustrated in FIG. 1, the TV is taken as 6 ml/kg/breath or 420 ml/breath, per this Example. The ADS for this exemplary adult male has an ADS volume of approximately 2 ml/kg/breath or approximately 140 ml/breath. Note that the ADS volume of the ADS is defined with the individual in non-distressed resting state, in certain aspects. Therefore, nearly ⅓ (e.g., 140/420) of each TV is parked within the ADS thereby contributing nothing to oxygenation. The alveolar TV is then (420 ml/breath-140 ml/breath) or 280 ml/breath, which represents the volume of oxygen being delivered to the alveolar region by each breath that actually contributes to oxygenation. In FIG. 1, the exemplary adult male has a respiratory rate (RR) of 10 breaths/min (e.g., 6 s/breath) so that the minute ventilation is (420 ml/breath) (10 breath/min) which is 4,200 ml/min. Assuming that the exhalation period is 1.5× the inhalation period, the resulting inhalation period is then 2.4 s and the exhalation period is 3.6 s. The alveolar minute ventilation is then (420 ml/breath-140 ml/breath) (10 breath/min) which is 2,800 ml/min and represents actual volume of respiratory gas being delivered each minute into the alveolar region where the oxygen can be absorbed. Note that the exemplary 70 kg adult male of Example 1 is breathing freely without assistance of any kind and is unencumbered by a facemask or other respiratory device of any kind.

### Example 2

Example 2 illustrates the impact of the ADS. In Example 2, the 70 kg adult male of Example 1 is suffering from hypoxia and has an increased respiratory rate of 20 breaths per minute while maintaining the same minute ventilation of 4200 ml/min (a conservative assumption). Again, the exemplary 70 kg adult male of Example 2 is breathing freely without assistance of any kind and is unencumbered by a facemask or other respiratory device. It is recognized that 20 breaths per minute may be a physically extreme condition but is used in this Example for purposes of explanation. The period for each breath is then 3 s with 1.2 s of inhalation and 1.8 s exhalation. The TV is then (4200 ml/min)/(20 breath/min) which is 210 ml/breath. The ADS remains 140 ml/breath, so that the alveolar TV becomes (210 ml/breath-140 ml/breath) which is 70 ml/breath, in contrast to the alveolar TV of 280 ml/breath per Example 1. The alveolar minute ventilation is then (70 ml/breath) (20 breath/min) which is 1,400 ml/min in contrast to the alveolar minute ventilation of 2,800 ml/min per Example 1. Thus, as illustrated by Example 2, the combination of the ADS with the increased RR decreases the delivery of oxygen to the alveolar regions of the lungs by 75%, with corresponding impact on hypoxia.

A facemask applied to the hypoxic adult male of Example 2 adds additional dead space volume (e.g., facemask volume of the facemask) within which no oxygen exchange takes place effectively adding to the ADS and further decreasing the alveolar minute ventilation, which is already low. In addition, exhaled CO₂ collects within the dead space volume from whence it may then be rebreathed thereby potentially creating, for example, hypercarbia and respiratory acidosis. Thus, it may be beneficial to decrease the dead space volume when providing respiratory gas in treatment of hypoxia.

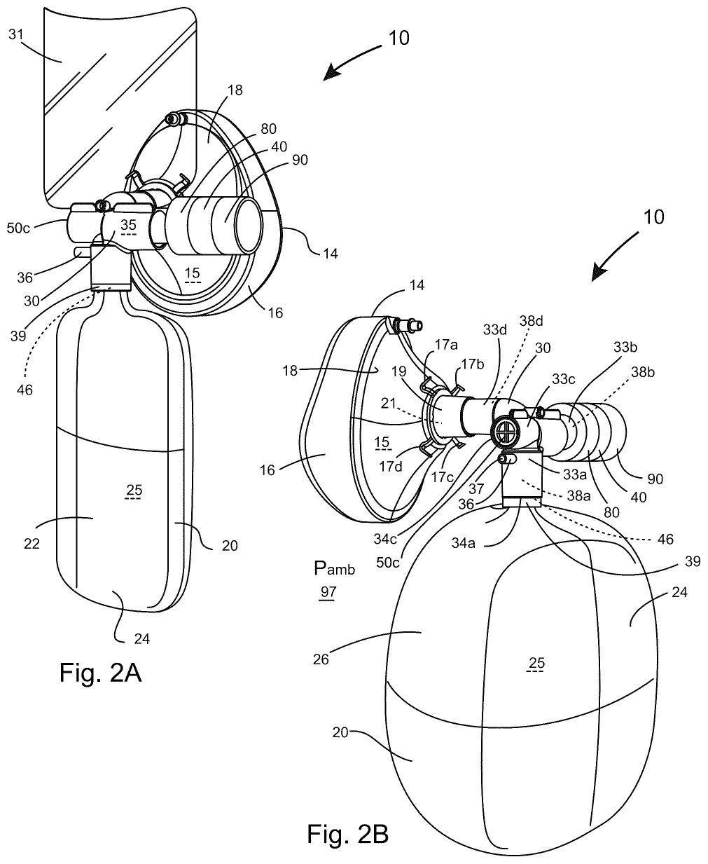

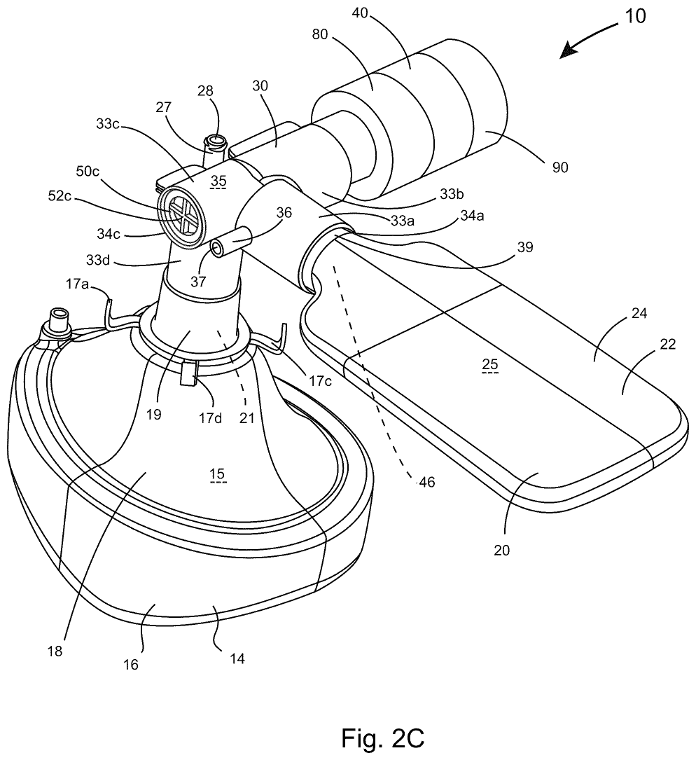

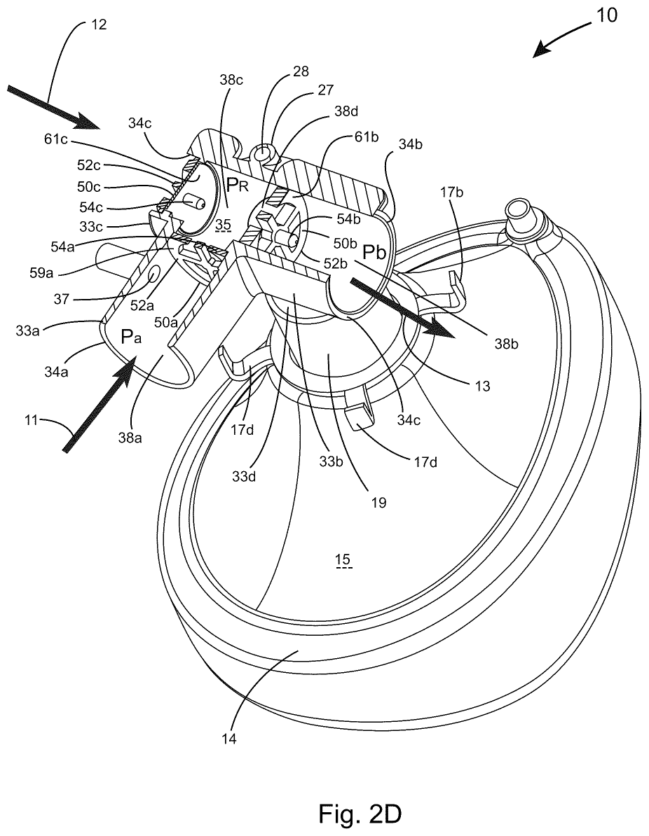

FIGS. 2A, 2B, 2C, 2D illustrate exemplary respiratory support apparatus 10 including manifold 30 pivotably secured to facemask 14. Facemask 14 includes cover 18 surrounded peripherally by cushion 16, and facemask conduit 19, which defines conduit passage 21, extends forth from cover 18, as illustrated. When secured to a user by head-strap(s) (not shown) engaged with head-strap hooks 17a, 17b, 17c, 17d, facemask 14 defines facemask chamber 15 over the user's nose and mouth. Manifold 30 control fluid communications with facemask chamber 15.

Cover 18 may be formed, for example, of rigid clear, polymer such as PVC, polyethylene terephthalate (PET), copolyester (such as Eastman Tritan®) or polycarbonate. Cushion 16 may be formed of soft polymer such as PVC or silicone. Cushion 16 may be adjustably inflatable, in various implementations. As illustrated in FIG. 2B, ambient environment 97 has ambient pressure p_{amb}.

Manifold 30 includes first arm 33a, second arm 33b, and third arm 33c generally in coplanar disposition in the form of a “Y” or “T” and fourth arm 33d generally normal to the coplanar disposition of first arm 33a, second arm 33b, and third arm 33c, in this implementation. As illustrated, second arm 33b is perpendicular to first arm 33a and third arm 33c is perpendicular to first arm 33 and axially aligned with second arm 33b (e.g., aligned at 180° with second arm 33b), which may enhance the grippability of manifold 30 thereby enabling manipulation of manifold 30, for example, for interchangeable engagement/disengagement of fourth arm 33d with facemask conduit 19 or with an endotracheal tube or laryngeal mask airway. A user may engage manifold 30 at the confluence of first arm 33a, second arm 33b, and third arm 33c with the palm while gripping first arm 33a, second arm 33b, and third arm 33c with the fingers. This illustrated alignment of first arm 33a, second arm 33b, and third arm 33c with fourth arm 33d may avoid interference with the patient and with caregivers moving about the patient particularly when facemask conduit 19 extends generally vertically from the face as first arm 33a, second arm 33b, and third arm 33c would be generally in parallel alignment with the face and not extending outward from the face where they might be bumped, etc. Thus, the alignment of first arm 33a, second arm 33b, and third arm 33c with fourth arm 33d illustrated in FIGS. 2A, 2B, 2C, 2D serves a functional purpose. Various other implementations may have other relational orientations of first arm 33a, second arm 33b, third arm 33c, and fourth arm 33d with respect to one another generally set at right angles in order to minimize a manifold volume of manifold chamber 35. Manifold 30 defines manifold chamber 35 and first arm 33a, second arm 33b, third arm 33c define arm passages 38a, 38b, 38c, respectively, that communicate fluidly with manifold chamber 35, as illustrated in FIG. 2D. Check valves 50a, 50b, 50c are positioned within arm passages 38a, 38b, 38c, respectively, to control fluid communications with manifold chamber 35 and, thus, with facemask chamber 15, as illustrated in FIG. 2D. Check valves 50a, 50b, 50c are held in position within arm passages 38a, b, c, d, respectively, by, for example, sonic welding, adhesive, compression fit, or capture by detent, as would be readily recognized by those of ordinary skill in the art upon study of this disclosure.

As illustrated in FIG. 2C, manifold 30 is rotatably secured in a fluid tight manner to facemask conduit 19 by the engagement of fourth arm 33d with facemask conduit 19 to allow fluid communication between manifold chamber 35 and facemask chamber 15 via arm passage 38d of fourth arm 33d and via conduit passage 21 of facemask conduit 19. For example, fourth arm 33d of manifold 30 may be secured to facemask conduit 19 of facemask 14 by interference fit. Fourth arm 33d and facemask conduit 19 are rigid so that manifold 30 is rigidly engaged with facemask 14, in this implementation. In other implementations, fourth arm 33d and/or facemask conduit 19 may be flexible. When secured to facemask 14, manifold 30 may be pivoted about fourth arm 33d as axis to position first arm 33a, second arm 33b, and third arm 33c in various orientations with respect to the user when facemask 14 is affixed to the user.

Facemask conduit 19 including conduit passage 21 may be of a standard size and standard configuration, such as those prescribed by ISO 5356-1: 2015 standards governing anesthesia masks and ventilation equipment. Fourth arm 33d may be sized and otherwise configured for secure engagement with facemask conduit 19 sized according to the ISO 5361:2023 standard. For example, fourth arm 33d may be sized to be insertably received for interference fit within conduit passage 21 of facemask conduit 19.

As illustrated in FIGS. 2A, 2B, 2C, bag 20 defines bag reservoir 25, and bag 20 is appended to bag conduit 39. Bag 20 is affixed to arm end 34a of first arm 33a of manifold 30, for example, by compression fit between bag conduit 39 and first arm 33a. Bag conduit 39 defines bag conduit passage 46 through which bag reservoir 25 fluidly communicates with arm passage 38a, as illustrated, to allow fluid communication between bag reservoir 25 and manifold chamber 35 of manifold 30. In FIG. 2A, bag 20 is illustrated in collapsed state 22, which may occur in later portions of user inhalation when respiratory gas 11 is generally withdrawn from bag reservoir 25. Bag 20 is illustrated in expanded state 26, in FIG. 2B, which may occur proximate completion of user exhalation when bag reservoir 25 is generally filled with respiratory gas 11. Bag 20 may be formed of compliant fluid-impermeable material such as polyethylene sheeting, and bag 20 may have display color 24 that enhances the visual apprehension of bag 20 to allow visual assessment of respiratory function. Display color 24 may be, for example, safety orange, safety red, safety green, or other bright neon color, pattern, or combination of color and pattern that aids a healthcare provider in visually perceiving bag 20 in collapsed state 22, expanded state 26, and as bag 20 transitions between collapsed state 22 and expanded state 26.

As illustrated in FIGS. 2C, 2D, inflow port 36 formed as a nipple on first arm 33a defines inflow passage 37. Tubing including various piping, hose(s), connector(s), and other fluid conveyances (not shown) for the conveyance of respiratory gas 11 may be received by inflow port 36 to fluidly communicate respiratory gas 11 via inflow passage 37 into arm passage 38a and thence into manifold chamber 35 through check valve 50a and into bag reservoir 25 through bag conduit 39.

As illustrated in FIG. 2D, check valve 50a is disposed within arm passage 38a of first arm 33a to control the flow of respiratory gas 11 through arm passage 38a into manifold chamber 35, and check valve 50b is disposed within arm passage 38b of second arm 33b to control the flow of outflow gas 13 from manifold chamber 35 through arm passage 38b and, thence, into the ambient environment. Check valve 50c is configured as an anti-asphyxiation valve and is disposed within arm passage 38c of third arm 33c to control inflow of ambient air 12 from ambient environment 97 into manifold chamber 35 in order to prevent asphyxia of the user should the flow of respiratory gas 11 be insufficient, as illustrated.

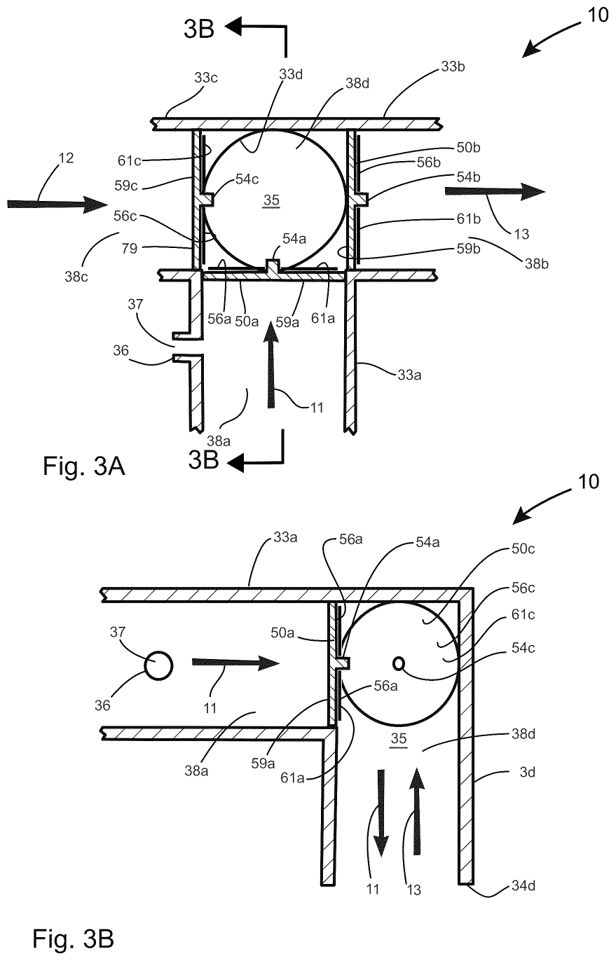

Manifold chamber 35 of manifold 30 is bounded by downstream side 61a of check valve 50a, upstream side 59b of check valve 50b, downstream side 61c of check valve 50c, and arm end 34d of fourth arm 33d and has an associated manifold volume of about 10 ml. Manifold 30, in this exemplary implementation, is configured to minimize the manifold volume of manifold chamber 35. Accordingly, downstream side 61a of check valve 50a, upstream side 59b of check valve 50b, and downstream side 61c of check valve 50c are positioned within arm passage 38a of first arm 33a, arm passage 38b of second arm 33b, and arm passage 38c of third arm 33c, respectively, concurrent with and tangential to a periphery of arm passage 38d of fourth arm 33d, as illustrated in FIG. 3A. Note that the proximity at which check valves 50a, 50b, 50c are placed to arm passage 38d may be limited by the manner in which check valves 50a, 50b, 50c are secured within arm passages 38a, 38b, 38c, respectively, but nonetheless check valves 50a, 50b, 50c are placed as close as practicable to the periphery of arm passage 38d. Second arm 33b and third arm 33c are perpendicular to first arm 33a, as illustrated. Thus, in certain implementations in accordance with the ISO 5356-1:2015 standard, check valves 50b, 50c are set apart from one another essentially by diameter 22 mm of passage 38d and each of check valves 50a, 50b, 50c has width 22 mm essentially equivalent to diameter 22 mm of passage 38d. Thus, the ability to minimize the manifold volume of manifold chamber 35 is limited by diameter 22 mm of passage 38d, and diameter 22 mm of passage 38d may be constrained by the ISO 5356-1:2015 standard. That is, diameter 22 mm of passage 38d may be stipulated by the ISO 5356-1:2015 standard. For example, outer diameter of fourth arm 33d is 2 mm in order to be insertably received within facemask conduit 19 that has inner diameter 2 mm. Fourth arm 33d has length 21 mm sufficient to engage securely facemask conduit 19. As a result, the manifold volume, for example, may be about 10 cm³. For a flow rate of 5 l/min (83.3 ml/s) of respiratory gas 11, a manifold detention time of manifold chamber 35 is less than about 0.12 s where the manifold detention time is given by the manifold volume divided by the flow rate of respiratory gas 11. Note that this manifold detention time of 0.12 s is an order of magnitude less than the exhalation period of 1.2 s per Example 2. Minimizing the manifold detention time of manifold chamber 35 facilitates flushing outflow gas 13 from the manifold chamber during exhalation, for example, during fourth operational state 98. Flushing of outflow gas 13 from the manifold chamber prevents rebreathing of the outflow gas 13 during subsequent inhalation(s).

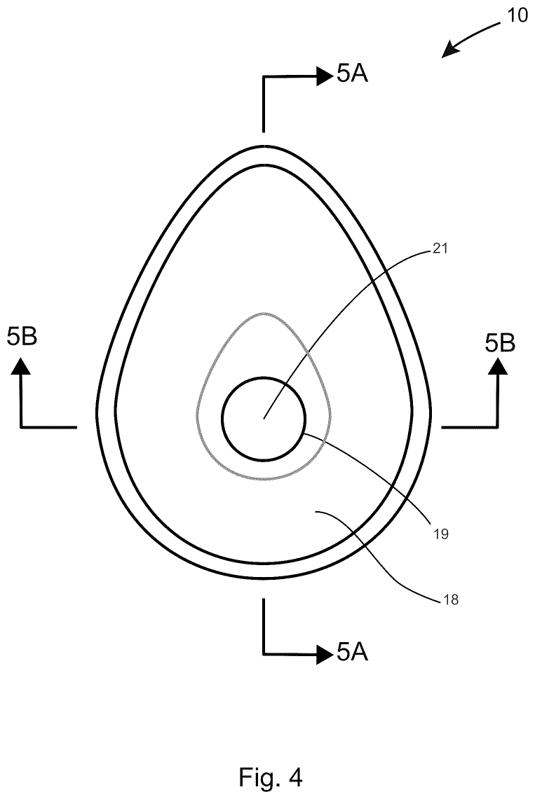

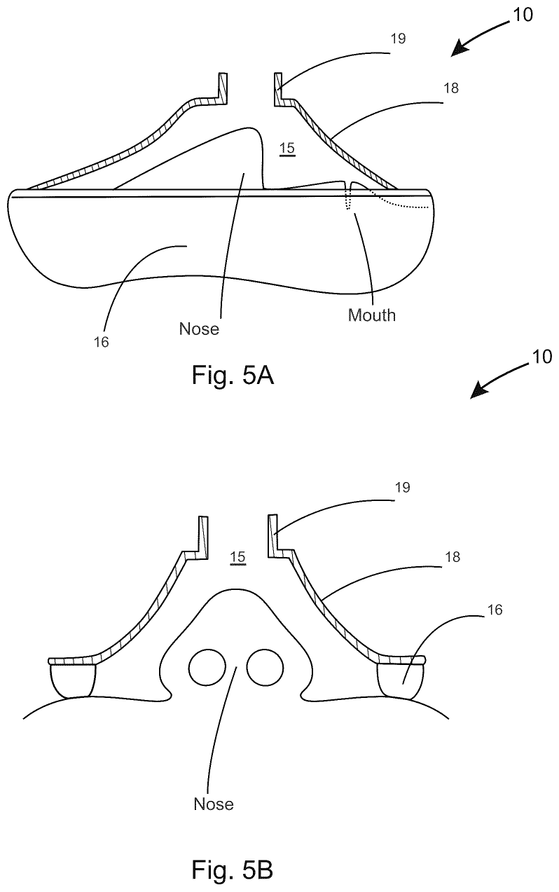

With reference to FIGS. 4, 5A, 5B, facemask 14 engaged with the user defines facemask chamber 15 bounded by cover 18, cushion 16, and the portions of the face of the user over which facemask 14 is engaged and having a reduced facemask volume by having a cone with a concave instead of a convex profile. Facemask volume further includes the volume of portions of facemask conduit not engaged with fourth arm 33d of manifold 30. Facemask 14, in this exemplary implementation, is configured to minimize the facemask volume of facemask chamber 15. For example, cover 18 of facemask 14 is contoured to accommodate the portions of the face of the user over which facemask 14 is engaged while minimizing the space between the portions of the face and cover 18. In various implementations, the facemask volume may be generally within a range of from about 50 cm³ to about 100 cm³ for a #5 adult mask and proportionally less for smaller masks. In various implementations, the facemask volume may be about 25 cm³ to about 150 cm³.

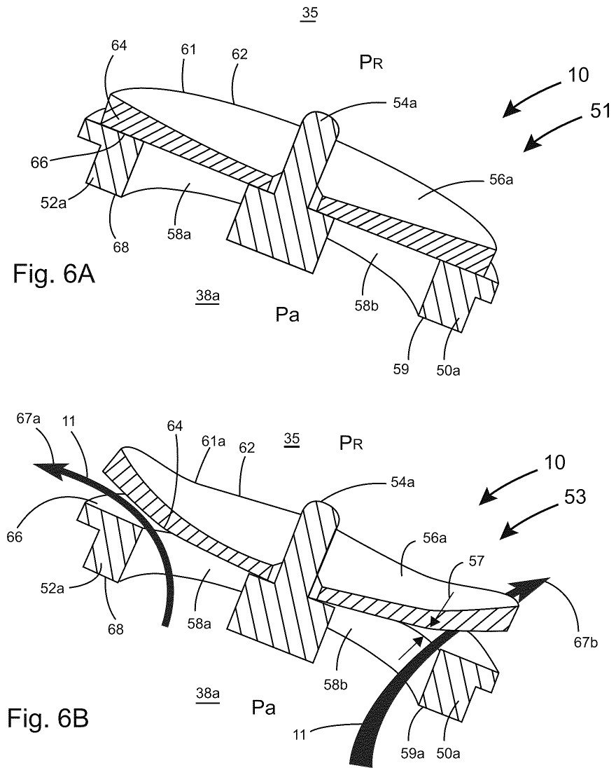

An exemplary implementation of check valve 50a is illustrated in FIGS. 6A, 6B. As illustrated, check valve 50a includes valve member 56a insertably received over pin 54a that extends forth from valve seat 52a to detain valve member 56a in cooperation with valve seat 52a. Check valves 50b, 50c that include pins 54b, 54c extending forth from valve seats 52b, 52c (see FIG. 2D) respectively, are illustrated as being formed similarly to check valve 50a, in this implementation. Thus, check valves 50b, 50c may operate similarly to check valve 50a, in this implementation. Of course, check valves 50a, 50b, 50c may variously differ from one another or may be otherwise configured, in various other implementations, as would be readily recognized by those of ordinary skill in the art upon study of this disclosure. Note that the valve members 56a, 56c of valves 50a, 50b, respectively, are omitted from FIG. 2D for purposes of clarity of explanation, as are monitoring package 40, PEEP valve 90, and anti-pathogen module 80.

As illustrated in FIGS. 6A, 6B, valve seat 52a outer perimeter of valve seat 52a may be secured within arm passage 38a of first arm 33a, as illustrated. Apertures, such as apertures 58a, 58b, formed in valve seat 52a allow gas flow through valve seat 52a, in this implementation. As illustrated, check valve 50a is oriented so that surface 62 of valve member 56a is on the downstream side 61a of check valve 50a and surface 68 of valve seat 52a is on the upstream side 59a of check valve 50a. That is, pins 54a, 54b, 54c are oriented to extend forth from valve seats 52a, 52b, 52c in a flow direction of respiratory gas 11, outflow gas 13, and ambient air 12, respectively, in this implementation.

Check valve 50a is positionable between closed position 51 illustrated in FIG. 6A and open position 53 illustrated in FIG. 6B. In closed position 51 illustrated in FIG. 6A, manifold pressure p_{R} within manifold chamber 35 on downstream side 61a of check valve 50a is greater than pressure pₐ within arm passage 38a on upstream side 59a of check valve 50a thereby holding portions of surface 64 of valve member 56a in biased engagement with portions of surface 66 of valve seat 52a. The biased sealing engagement of portions of surface 64 with portions of surface 66 sealingly engages valve member with valve seat 52a thus blocking gas flow through check valve 50a from downstream side 61 (e.g., manifold chamber 35) to upstream side 59 (e.g., arm passage 38a), in this implementation.

In open position 53 illustrated in FIG. 6B, pressure pₐ within arm passage 38a on upstream side 59a of check valve 50a is greater than manifold pressure p_{R} within manifold chamber 35 on downstream side 61a of check valve 50a thereby causing portions of surface 64 of valve member 56a to flex into spaced relation with portions of surface 66 of valve seat 52a, in this implementation. In open position 53, portions of surface 64 are in spaced relation with portions of surface 66, and respiratory gas 11 flows through check valve 50a from upstream side 59a (e.g., arm passage 38a) to downstream side 61a (e.g., manifold chamber 35) by flowing through apertures, such as apertures 58a, 58b, in valve seat 52a and through gap 57 between portions of surface 64 of valve member 56a and portions of surface 66 of valve seat 52a, as indicated by arrows 67a, 67b in FIG. 4B.

Valve seats 52a, 52b, 52c may be made of hard plastic, and valve members 56a, 56b, 56c may be made of a soft, flexible material such as rubber or silicone having a durometer value (e.g., Shore hardness) within a selected range of durometer values that enable proper operation of the valve. If, for example, the durometer value of the valve member, such as valve member 56c, is too low (e.g., valve member 56c is too soft), anti-asphyxiation check valve 50c may open prematurely, thereby introducing ambient air 12 that dilutes respiratory gas 11 in manifold chamber 35, and, thus, within facemask chamber 15, as the user in inhaling into alveolar regions of the lungs. Such dilution of respiratory gas 11 reduces oxygen delivery to the user thereby reducing the effectiveness of treatment of hypoxia. If the durometer value of valve member 56c is too high (e.g., valve member 56c is too hard), anti-asphyxiation valve 56c may fail to open adequately, which increases work of breathing for the user. That is, anti-asphyxiation check valve 50c fails to function if the durometer value of valve member 56c is too high. In various implementations, valve member 56c has a Shore hardness within a range of from about 25 to about 80 on the 00 scale. In various aspects, valve member 56c has a Shore hardness within a range of from about 40 to about 60 on the 00 scale. In various aspects, valve member 56c has a Shore hardness of about 60 on the 00 scale. Valve members 56a, 56b in various implementations may have Shore hardness somewhat less than the Shore hardness of valve member 56c in order to ensure particularly that check valve 50a opens before 50c. The Shore hardness of valve members 56a, 56b may be selected to ensure proper operation of check valves 50a, 50b. At least portions of manifold 30 including first arm 33a, second arm 33b, third arm 33c, and fourth arm 33d at least portions of check valves 50a, 50b, and at least portions of anti-asphyxiation check valve 50c may be formed of various suitable plastics, for example, by 3-D printing including other reproduction or additive technologies that may facilitate manufacture including manufacture in situ. Valve members 56a, 56b, 56c may be variously formed of polysiloxane and other silicone polymers including, for example, dimethicone, cyclopentasiloxane, dimethiconol, phenyl trimethicone, amodimethicone, and cyclomethicone.

Exemplary respiratory support apparatus 10 includes shield 31 that is attached to manifold 30 to form a barrier, for example, against infectious aerosol that may otherwise be directed at the user's eyes, as illustrated in FIG. 2A. Note that shield 31 is omitted from the other Figures for purposes of clarity of explanation. Shield 31 may be formed of a transparent material such as acrylic sheet. Shield 31 is optional and may be omitted, in other implementations.

Exemplary respiratory support apparatus 10 includes anti-pathogen module 80, monitoring package, 40, and PEEP valve 90, as illustrated in FIGS. 2A, 2B, 2C. Anti-pathogen module 80 is received by second arm 33b of manifold 30 in fluid communication with manifold chamber 35 of manifold 30 to remove pathogens from outflow gas 13, as illustrated. Pathogens, as used herein, may include, for example, viruses, bacteria, and fungi, as well as bodily fluids and various noxious, odiferous, or undesirable substances as may be included in outflow gas 13. Anti-pathogen module 80 may be omitted, in some implementations. Anti-pathogen module 80 may include various filter(s), activated carbon, and disinfectant(s), as would be readily recognized by those of ordinary skill in the art upon study of this disclosure.

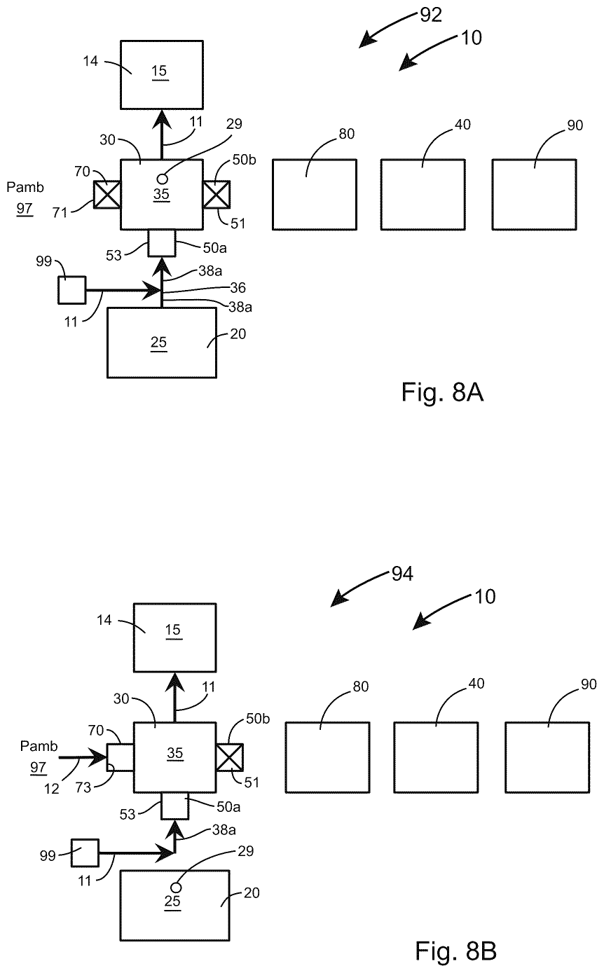

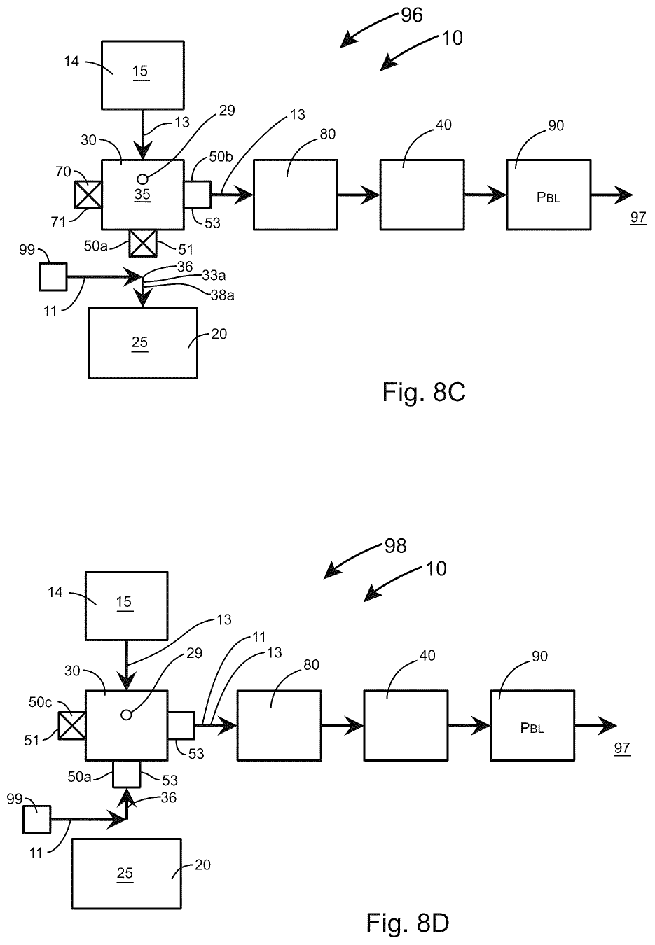

Monitoring package 40, as illustrated in FIGS. 2A, 2B, 2C, is secured to antipathogen module 80 in fluid communication with manifold chamber 35 to monitor attribute 44 of the outflow gas 13, as illustrated. Monitoring package 40 may be omitted, in some implementations. As illustrated in FIGS. 2C, 2D, sensor port 27 on manifold 30 defines sensor passage 28 that communicates through manifold 30 with manifold chamber 35. Sensor 29 (see FIGS. 8A, 8B, 8C, 8D) may communicate through sensor passage 28 with manifold chamber 35 and/or facemask chamber 15 to monitor an attribute, such as attribute 44, within manifold chamber 35 and/or within facemask chamber 15. Attribute 44, which may be monitored by sensor 29 and/or monitoring package 40, may include, for example, EtCO₂ (end tidal carbon dioxide indicative of adequacy of ventilation), F_{E}NO (exhaled nitric oxide indicative of airway inflammation, pulmonary hypertension and cardiac failure) or other metabolic gases such as ketones in diabetic ketoacidosis, carbon monoxide, or core temperature. Attribute 44 may include changes in breathing cycle that, for example, may indicate hypopnea, and attribute 44 may indicate loss of pressure that may be indicative of apnea or a loose facemask. Monitoring package 40 and/or sensor 29 may be configured for digital control and may communicate via various wired or wireless technologies, in various implementations.

PEEP valve 90, as illustrated in FIGS. 2A, 2B, 2C, may optionally be positioned downstream of monitoring package 40 in fluid communication with manifold chamber 35 of manifold 30 to maintain a selected baseline pressure p_{BL} within manifold chamber 35 as the user exhales. Baseline pressure p_{BL} may be selected in order to maintain pressure on the most distal airways sufficient to prevents alveoli from collapsing during exhalation. Alveoli collapse may occur normally from absorption of oxygen in the alveolar sacs, and, unless these sacs are distended open, a ventilation perfusion mismatch and shunting develop resulting in loss of gas exchange ability. In ARDS (acute respiratory distress syndrome), loss of lung compliance may necessitate the use of PEEP valve 90 to improve oxygenation. PEEP valve 90 may be adjusted, for example, between 5 cm to 25 cm of water to set correspondingly the selected baseline pressure p_{BL}, as would be readily recognized by those of ordinary skill in the art upon study of this disclosure. PEEP valve 90 may be manufactured, for example, by Becton Dickinson and Company of Franklin Lakes, NJ, Ambu A/S of Denmark, or Besmed of New Taipei City, Taiwan.

In exemplary respiratory support apparatus 10, outflow gas 13 passes from manifold chamber 35 through anti-pathogen module 80, then through monitoring package 40, followed by passage through PEEP valve 90, and is discharged into the ambient environment from PEEP valve 90. Anti-pathogen module 80, monitoring package 40, and PEEP valve 90 may be arranged in other orders with respect to the flow of outflow gas 13, in various other implementations. Anti-pathogen module 80, monitoring package 40, and PEEP valve 90 are all optional, and, thus, may or may not be included, in various implementations.

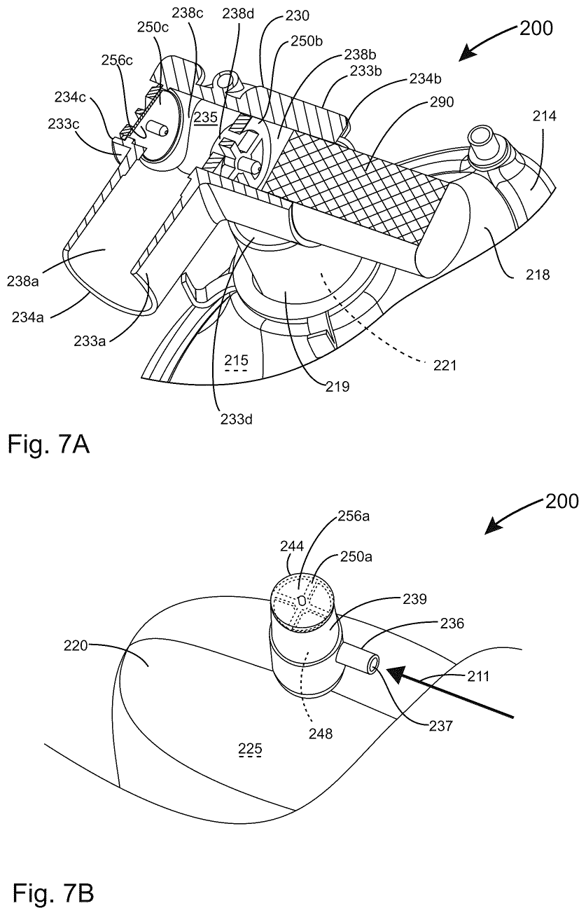

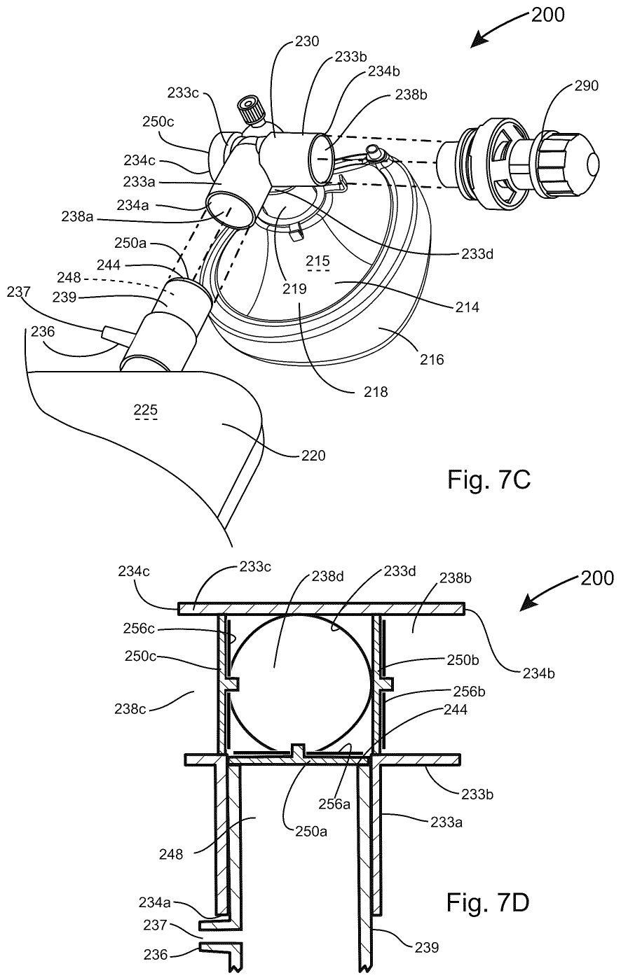

FIGS. 7A, 7B, 7C, 7D illustrate exemplary respiratory support apparatus 200 including manifold 230 secured to facemask 214. As illustrated, facemask 214 includes cover 218 surrounded peripherally by cushion 216, and facemask conduit 219, which defines conduit passage 221, extends forth from cover 218. When secured to the user, facemask 214 defines facemask chamber 215 over the user's nose and mouth.

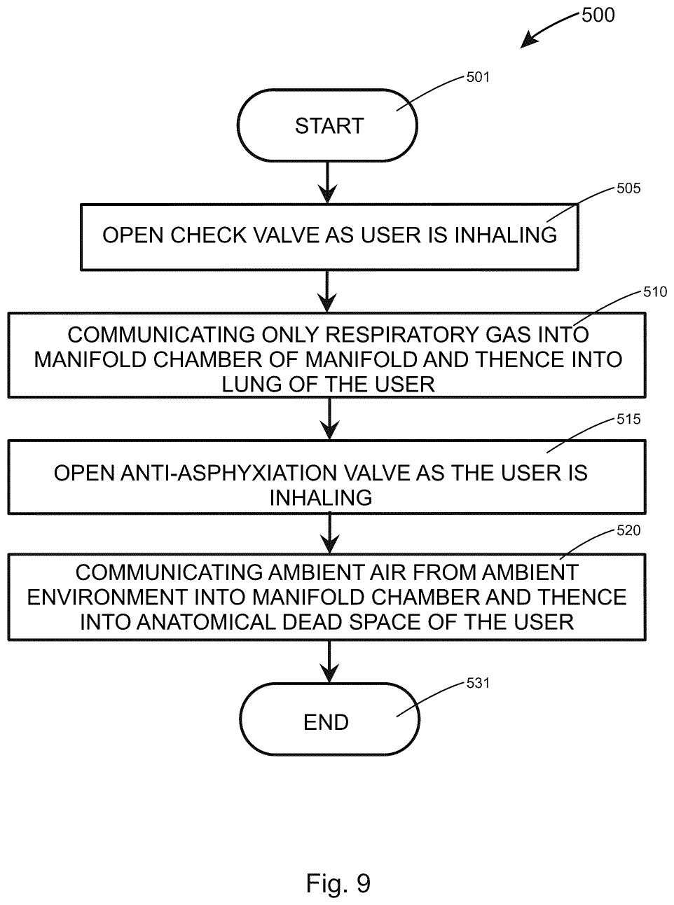

Manifold 230 includes first arm 233a, second arm 233b, and third arm 233c generally in coplanar disposition in the form of a “Y” or “T” and fourth arm 233d generally normal to the coplanar disposition of first arm 233a, second arm 233b, and third arm 233c, as illustrated. Manifold 230 defines manifold chamber 235 and first arm 233a, second arm 233b, third arm 233c, and fourth arm 233d define arm passages 238a, 238b, 238c, 238d, respectively, that communicate fluidly with manifold chamber 235, as illustrated in FIG. 9. Fourth arm 233d may either insertably receive portions of facemask conduit 219 within arm passage 238d or portions of fourth arm 233d may be received within conduit passage 221 fo facemask conduit 219 to secure manifold 230 to facemask 214 by interference fit so that facemask chamber 215 is in fluid communication with manifold chamber 235, in various implementations.

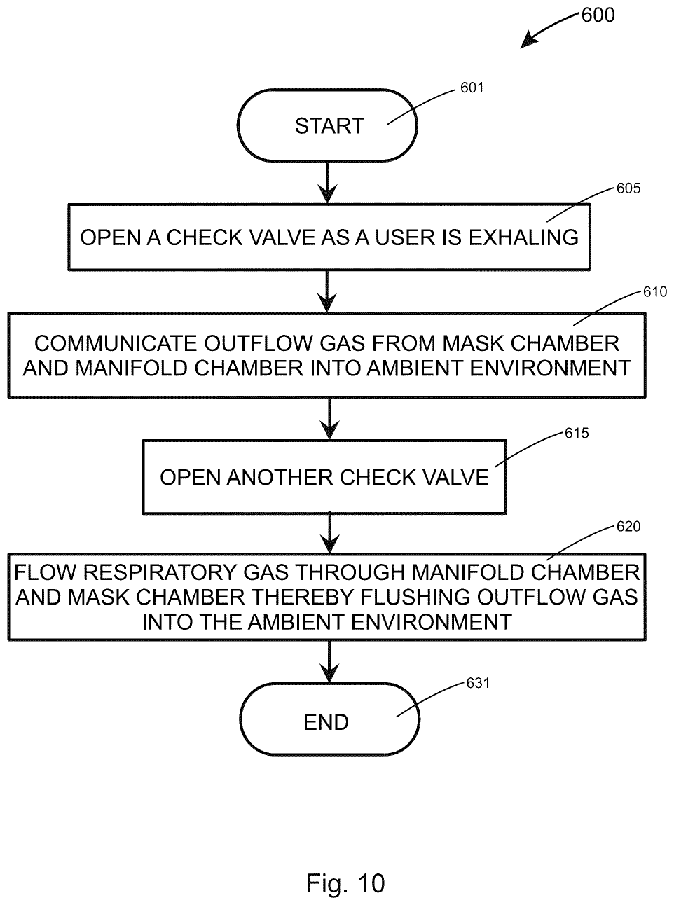

As illustrated in FIG. 7B, bag 220 defines bag reservoir 225. Bag 220 is appended to bag conduit 239 that defines bag conduit passage 248 through which bag reservoir 225 fluidly communicates, as illustrated. Check valve 250a is received within bag conduit passage 248 proximate bag conduit end 244 opposite of bag 220, as illustrated in FIG. 10. Bag conduit end 244 and portions of bag conduit 239 may then be insertably received within arm passage 238a of first arm 233a to position check valve 250a within arm passage 238a, as illustrated in FIGS. 7C, 7D. The portions of bag conduit 239 may be held within arm passage 238a by interference fit, for example. This contrasts with exemplary respiratory therapy apparatus 10 wherein check valve 50a is secured within passage 38a separable from bag conduit 39. Note that check valve 250a is positioned at the circumference of arm passage 238d with bag conduit 239 received within arm passage 238a of fourth arm 233d, as illustrated in FIG. 7D. Also, check valves 250b, 250c are positioned at the circumference of arm passage 238d of fourth arm 233d, as illustrated in FIG. 7D. Check valves 250a, 250b, 250c, as illustrated in FIG. 7D, include valve members 256a, 256b, 256c, respectively. The positioning of check valves 250a, 250b, 250c at the circumference of arm passage 238d as illustrated in FIG. 7D, as opposed to elsewhere within arm passages 238a, 238b, 238c of arms 233a, 233b, 233c, minimizes the manifold volume of manifold chamber 235 thereby minimizing the manifold detention time of manifold chamber 235.

When so received within arm passage 238a, check valve 250a controls fluid communication of respiratory gas 211 from inflow passage 237 and bag reservoir 225 with manifold chamber 235. As illustrated in FIGS. 7B, 7C, inflow port 236, which is formed as a nipple on bag conduit 239, defines inflow passage 237 through which respiratory gas 211 may be communicated with bag conduit passage 248 and thence into bag reservoir 225. Respiratory gas 211 may be communicated into arm passage 238a from bag reservoir 225 through bag conduit passage 248 and, thence, into manifold chamber 235 as controlled by check valve 250a.

Thus, in exemplary respiratory support apparatus 200, check valves 250a, 250b and anti-asphyxiation check valve 250c are disposed within arm passages 238a, 238b, 238c, respectively, to control fluid communications with manifold chamber 235 and, thus, with facemask chamber 215. As illustrated in FIG. 7C, PEEP valve 290 may be insertably received within arm passage 238b at arm end 234b downstream of check valve 250b for securement, for example, by interference fit. PEEP valve 290 may be omitted in certain implementations.

In exemplary operations of a respiratory support apparatus, such as respiratory support apparatus 10, 200, a facemask, such as facemask 14, 214, may be secured to a user to define facemask chamber, such as facemask chamber 15, 215 that encloses the user's nose and mouth so that the user inhales from the facemask chamber and exhales into the facemask chamber. A manifold, such as manifold 30, 230, may be secured to facemask 14, and the manifold may variously include a PEEP valve, such as PEEP valve 90, 290, anti-pathogen module, such as anti-pathogen module 80, and/or a monitoring package, such as monitoring package 40. The PEEP valve may be configured to set the selected baseline pressure p_{BL} within a manifold chamber, such as manifold chamber 35, 235, of the manifold, and, thus, within the facemask chamber and within the user's lungs as the user exhales. A bag, such as bag 20, 220, that defines a bag reservoir, such as bag reservoir 25, 225, may be in fluid communication with the manifold chamber of the manifold. A respiratory gas, such as respiratory gas 11, 211, may be communicated with the manifold via an inflow port, such as inflow port 36, 236.

The facemask, the manifold, and the bag may be provided as separate elements that may be joined together, for example, by interference fit, in various implementations. For example, a facemask conduit, such as facemask conduit 19, 219, of the facemask may be engaged with a fourth arm, such as fourth arm 33d, 233d, of the manifold by interference fit to the secure facemask to the manifold. A bag conduit, such as bag conduit 39, 239, of the bag may be engaged with a first arm, such as first arm 33a, 233a of the manifold by interference fit to secure the bag to the manifold. The PEEP valve, monitoring package, and/or anti-pathogen module, if any, may be secured to one another or to a second arm, such as second arm 33b, 233b, by interference fit. Various guideways, keyways, stops, Luer lock fittings, conduits, and so forth may be included with the respiratory support apparatus that enable joining of facemask, manifold, bag, PEEP valve, monitoring package, and anti-pathogen module, as well as enabling fluid communication of the inflow port with a gas source, such as gas source 99 (see FIGS. 8A, 8B, 8C, 8D), as would be readily understood by those of ordinary skill in the art upon study of this disclosure.

FIGS. 8A, 8B, 8C, 8D illustrate operations of exemplary respiratory support apparatus 10. Exemplary respiratory support apparatus 200 may operate similarly to respiratory support apparatus 10. The respiratory gas 11 is communicated into arm passage 38a of first arm 33a through inflow passage 37 of inflow port 36 from gas source 99, as illustrated in FIGS. 8A, 8B, 8C, 8D. Gas source 99 may be, for example, a cylinder of compressed gas or mains gas. In various implementations, gas source 99 may include an oxygen concentrator. The oxygen concentrator may, for example, supply 85-94% oxygen as respiratory gas 11 at a continuous flow of 5-10 L/min. Gas source 99 may include a pressure manifold that allows regulating of pressure pₐ within arm passage 38a of first arm 33a, for example, when check valve 50a is in closed position 51. In various implementations, gas source 99 may supply 85-94% oxygen as respiratory gas 11 at a continuous flow between about 2 L/min and about 15 L/min. In various implementations, gas source 99 may supply 85-94% oxygen as respiratory gas 11 at a continuous flow of around 10 L/min. The gas source 99 may be located in other than a hospital setting (e.g., a home or residential setting), in certain implementations.

As illustrated in FIGS. 8A, 8B, 8C, 8D, exemplary respiratory support apparatus 10 may operate in exemplary first operational state 92, in exemplary second operational state 94, in exemplary third operational state 96, and in exemplary fourth operational state 98, respectively, and may transition between first operational state 92, second operational state 94, third operational state 96, and fourth operational state 98. First operational state 92, second operational state 94, third operational state 96, and fourth operational state 98 may be configured to cooperate in order to maximize the delivery of respiratory gas to the alveolar regions. Respiratory support apparatus 10 transitions between first operational state 92, second operational state 94, third operational state 96, and fourth operational state 98 as prompted by the user's spontaneous inhalation and exhalation. For example, as the user inhales, respiratory support apparatus 10 may operate in first operational state 92 illustrated in FIG. 8A, and, respiratory support apparatus 10 may operates in second operational state 94 illustrated in FIG. 8B. Second operational state 94, illustrated in FIG. 8B, prevents suffocation of the user from insufficient respiratory gas 11, or allows for operation with limited supply of respiratory gas 11 from gas source 99. Thus, second operational state 94 may be entered from first operational state 92 as the user inhales with the user first inhaling respiratory gas 11 in first operational state 92 and then inhaling respiratory gas 11 mixed with ambient air 12 in second operational state 94. Ambient air may predominate in second operational state 94.

Note that, in the illustrated implementations of FIGS. 8A, 8B, 8C, check valves 50a, 50b, 50c are positioned between closed position 51 and open position 53 solely by the user's spontaneous breathing without assistance, for example, of electromechanical devices such as solenoid(s). Pressure differences between manifold pressure p_{R} within manifold chamber 35, pressure pₐ within arm passage 38a and pressure p_{b} within arm passage 38b position check valves 50a, 50b, respectively, between closed position 51 and open position 53, in various implementations. Pressure differences between manifold pressure p_{R} within manifold chamber 35 and ambient pressure p_{amb} in ambient environment 97 position check valve 50c between closed position 51 and open position 53, in various implementations. Thus, for example, no power source of electrical power is required to position check valves 50a, 50b, 50c respectively, between closed position 51 and open position 53 thereby transitioning respiratory support apparatus 10 between first operational state 92, second operational state 94, third operational state 96, and fourth operational state 98.

As the user inhales, respiratory support apparatus 10 operates in first operational state 92, as illustrated in FIG. 8A. As illustrated in FIG. 8A, respiratory gas 11 from gas source 99 flows into arm passage 38a of first arm 33a via inflow port 36 and respiratory gas 11 flows into arm passage 38a of first arm 33a from bag reservoir 25 of bag 20, in first operational state 92. Respiratory gas 11 is withdrawn from bag reservoir 25 into arm passage 38a during first operational state 92 thereby augmenting the flow of respiratory gas 11 from gas source 99 in order to provide sufficient respiratory gas 11 including sufficient respiratory gas 11 necessary to meet the peak inspiratory flow rate (PIFR) for inhalation by the user. Although the PIFR of 1000 ml/second, for example, lasts only briefly, having an adequate reserve of respiratory gas to meet peak inspiratory flow demand is crucial for delivering respiratory gas into the alveolar region of the lungs. Bag 20 is in expanded state 26 as first operational state 92 is initiated, and bag 20 is in collapsed state 22 when respiratory support apparatus 10 completes first operational state 92 due to withdrawal of respiratory gas 11 from bag reservoir 25 during first operational state 92.

As the user inhales, manifold pressure p_{R} within manifold chamber 35 decreases to less than pressure pₐ within arm passage 38a (e.g., pₐ>p_{R}) thereby placing check valve 50a in open position 53, and manifold pressure p_{R} within manifold chamber 35 decreases to less than pressure p_{b} within arm passage 38b (e.g., p_{b}>p_{R}) thereby placing check valve 50b in closed position 51. Check valve 50a in open position 53 allows respiratory gas 11 to flow from arm passage 38a through check valve 50a into manifold chamber 35 of manifold 30. Respiratory gas 11 then flows from manifold chamber 35 into facemask chamber 15 of facemask 14 for inhalation by the user.

Because check valve 50b is in closed position 51 in first operational state 92, there is no flow of respiratory gas 11 from manifold chamber 35 through check valve 50b into arm passage 38b of second arm 33b. In first operational state 92, flow of respiratory gas 11 into manifold chamber 35 maintains manifold pressure p_{R} within manifold chamber 35 greater than pressure pₓ where pressure pₓ may be less than or equal to ambient pressure p_{amb} in ambient environment 97 to maintain check valve 50c in closed position 51. Thus, there is no flow of ambient air 12 through check valve 50c into manifold chamber 35, in first operational state 92.

The user is inhaling without sufficient respiratory gas 11 being available for the user to inhale in second operational state 94, as illustrated in FIG. 8B. For example, in second operational state 94, there is insufficient inflow of respiratory gas 11 to meet all of inspiratory demand and essentially no respiratory gas 11 is available in bag reservoir (e.g., bag reservoir 25 is in collapsed state 22 having been depleted during first operational state 92) so that supplementation with ambient air 12 is necessary for the user to complete a breath. In second operational state 94, manifold pressure p_{R} within manifold chamber 35 has decreased to sufficiently less than ambient pressure p_{amb} (e.g., p_{R}<p_{amb}) due to user inhalation thereby positioning check valve 50c into open position 53, as illustrated. Check valve 50c in open position 53 allows ambient air 12 to flow into manifold chamber 35 from ambient environment 97 and, thence, into facemask chamber 15 of facemask 14 for inhalation by the user, as illustrated in FIG. 8B, thereby functioning as an anti-asphyxiation valve. Check valve 50b is in closed position 51 to prevent ambient air 12 from flowing from manifold chamber 35 into arm passage 38b, respectively, during second operational state 94, as illustrated. Although check valve 50a is illustrated in open position 53, check valve 50a may be in closed position 51, or check valve 50a may fluctuate between open position 53 and closed position 51 during second operational state 94. Note that second operational state 94 may provide a safety measure that prevents suffocation of the user in the event the flow of respiratory gas 11 is interrupted or terminated, for example, due to human error or equipment failure.

Second operational state 94 may be entered during an inhalation following first operational state 92 if a quantity of respiratory gas 11 available is less than the tidal volume of the user. In such situations, the user draws respiratory gas into the lungs until the entire available quantity of respiratory gas 11 is drawn into the lungs. Continued inhalation then decreases manifold pressure p_{R} within manifold chamber 35 to less than pressure pₓ below ambient pressure p_{amb} (e.g., p_{R}≤pₓ<p_{amb}) thus positioning anti-asphyxiation check valve 50c in open position 53 thereby providing ambient air 12 to the user that may be in addition to respiratory gas 11 that may continue flowing through check valve 50a. That is, check valve 50c opens at a pressure at least pₓ below ambient pressure p_{amb}. Check valve 50c may be configured to open so that ambient air 12 is inhaled proximate the end of an inhalation so that ambient air 12 fills the ADS thereby driving respiratory gas 11 deeper into the alveolar regions of the lungs. Thus, oxygen as respiratory gas 11 may be infused into the user through the alveolar regions while the anatomical dead space of the respiratory system that does not absorb oxygen is filled with ambient air 12.

For example, valve member 56c may be configured by, for example, configuring the durometer value of valve member 56c, the thickness of valve member 56c, and the change in thickness radially of valve member 56c (if any) so that anti-asphyxiation check valve 50c opens from closed position 51 to open position 53 later during inspiration so that respiratory gas 11 is communicated into the alveolar region initially during inspiration (e.g., first operational state 92) and, if necessary, respiratory gas 11 diluted with ambient air 12 is communicated generally into the anatomical dead space, not into alveolar region, during later portions of inspiration (e.g., second operational state 94).

As the user exhales, respiratory support apparatus 10 operates in third operational state 96, as illustrated in FIG. 8C. In third operational state 96, manifold pressure p_{R} within manifold chamber 35 is greater than pressure p_{b} within arm passage 38b (e.g., p_{R}>p_{b}) due to user exhalation, thereby placing check valve 50b in open position 53, and manifold pressure p_{R} within manifold chamber 35 is greater than pressure pₐ within arm passage 38a (e.g., p_{R}>pₐ), thereby placing check valve 50a in closed position 51, as illustrated.

With check valve 50b in open position 53, outflow gas 13, which comprises exhalation from the user including CO₂, flows from facemask chamber 15 into manifold chamber 35, flows from manifold chamber 35 through check valve 50b into arm passage 38b of second arm 33b. Outflow gas 13 then flows through arm passage 38b for discharge to ambient environment 97. As illustrated, outflow gas 13 flows successively from arm passage 38b through anti-pathogen module 80, through monitoring package 40, and through PEEP valve 90. Pathogens may be removed from outflow gas 13 by anti-pathogen module 80. Attribute 44 of outflow gas 13 may be detected by monitoring package 40, and the monitoring package may communicate data indicative of attribute 44 to a computer. Outflow gas 13 is discharged into ambient environment 97 from PEEP valve 90, as illustrated. Anti-pathogen module 80, monitoring package 40, and PEEP valve 90 may be disposed in various sequences so that outflow gas 13 may flow in various sequences through anti-pathogen module 80, monitoring package 40, and PEEP valve 90, in various other implementations. Any or all of anti-pathogen module 80, monitoring package 40, and PEEP valve 90 may be omitted, in various other implementations.

During third operational state 96, respiratory gas 11 flows into arm passage 38a of first arm 33a and thence into bag reservoir 25 of bag 20 to replenish respiratory gas 11 within bag reservoir 25, as illustrated in FIG. 8B. Because check valve 50a is in closed position 51 in third operational state 96, there is no flow of respiratory gas 11 from arm passage 38a of first arm 33a into manifold chamber 35. All of the respiratory gas 11 flows into bag reservoir 25. Bag 20, which may be in collapsed state 22 at the initiation of second operational state 94, may be in expanded state 26 at the completion of second operational state 94.

The manifold pressure p_{R} is sufficient to maintain check valve 50c that functions as an anti-asphyxiation valve in closed position 51 in third operational state 96. Thus, as illustrated, there is no flow of ambient air 12 through anti-asphyxiation check valve 50c from ambient environment 97 into manifold chamber 35 in third operational state 96.

Sensor 29, as illustrated in FIGS. 8A, 8B, 8C, 8D, communicates with manifold chamber 35 via sensor passage 29 to sense attribute 44 of outflow gas 13. Outflow gas 13 should be undiluted in order that attribute 44 is sensed accurately by sensor 29. Because there is no flow of respiratory gas 11 into manifold chamber 35 nor is there any flow of ambient air 12 into manifold chamber 35, outflow gas 13 in manifold chamber 35 is generally undiluted thereby allowing for accurate measurement of attribute 44 by sensor 29 during third operational state 96.

It should be noted that PEEP valve 90 sets baseline pressure p_{BL} within manifold chamber 35 during user exhalation that is greater than ambient pressure p_{amb}, in implementations that includes PEEP valve 90. For example, baseline pressure p_{BL} may be within a range of from about 5 mm H₂O to about 25 mm H₂O. Manifold pressure p_{R} within manifold chamber 35 and pressures pₐ, p_{b} within arm passages 38a, 38b, respectively, may fluctuate with respect to baseline pressure p_{BL} and with respect to ambient pressure p_{amb} as the user inhales and exhales and check valves 50a, 50b are positioned between open position 51 and closed position 53.

As the user continues to exhale and bag 20 generally reaches expanded state 26 from inflow of respiratory gas 11, respiratory support apparatus 10 transitions from third operational state 96 into fourth operational state 98, which is illustrated in FIG. 8D. In fourth operational state 98, pressure pₐ within arm passage 38a is greater than manifold pressure p_{R} within manifold chamber 35 (e.g., pₐ>p_{R}), thereby placing check valve 50a in open position 53, and manifold pressure p_{R} within manifold chamber 35 is greater than pressure p_{b} within arm passage 38b (e.g., p_{R}>p_{b}) due to user exhalation and pressure pₐ within arm passage 38a, thereby placing check valve 50b in open position 53, as illustrated. Thus, in general, the filling of bag reservoir 25 of bag 20 during exhalation shifts respiratory support apparatus 10 from third operational state 96 to fourth operational state 98. Note that check valve 50c is in closed position 51 in fourth operational state 98, so that there is no flow of ambient air 12 through anti-asphyxiation check valve 50c from ambient environment 97 into manifold chamber 35.

In fourth operational state 98, respiratory gas 11 flows from arm passage 38a through check valve 50a and thence through manifold chamber 35 and facemask chamber 15 where the respiratory gas 11 is entrained with outflow gas 13, and the combined respiratory gas 11 and outflow gas 13 then flows through check valve 50b for discharge to ambient environment 97. This flow of respiratory gas 11 through manifold chamber 35 and facemask chamber 15 and thence into ambient environment 97 flushes outflow gas 13 from manifold chamber 35 and facemask chamber 15 while replenishing manifold chamber 35 and facemask chamber 15 with respiratory gas 11 thereby decreasing a quantity of the outflow gas 13 within manifold chamber 35 and facemask chamber 15. Thus, the user then generally inhales respiratory gas 11 or a respiratory gas 11 outflow gas 13 mixture, not solely outflow gas 13, from facemask chamber 15 and from manifold chamber 35 generally immediately upon beginning of inhalation as per operational state 92 thereby providing respiratory gas 11 to the alveolar regions of the lungs. Because there is flow of respiratory gas 11 through manifold chamber 35, attribute 44 is not measured by sensor 29 during fourth operational state 98.

Dead space volume may be defined as the manifold volume of manifold chamber 35 plus the facemask volume of the facemask chamber 15. A detention time t_{D} may be defined as the dead space volume divided by the flow rate of respiratory gas 11 passing through the dead space volume from check valve 50a through check valve 50b during fourth operational state 98. Minimizing detention time t_{D} by minimizing the dead space volume of respiratory support apparatus 10 increases the flushing of dead space volume during fourth operational state 98. Increasing the flow rate of respiratory gas 11 decreases the detention time t_{D}. The smaller the detention time t_{D}, the more flushing of outflow gas 13 from the dead space volume will occur for a given flow rate of respiratory gas 11 for a given time. The dead space volume may be minimizing by minimizing the manifold volume of manifold chamber 35, by minimizing the facemask volume of the facemask chamber 15, or by minimizing both the manifold volume of manifold chamber 35 and the facemask volume of the facemask chamber 15, in various implementations. Accordingly, minimizing the dead space volume may be an important consideration in the design of the respiratory support apparatus, such as exemplary respiratory support apparatus 10, 200. Furthermore, the bag volume of the bag reservoir, such as bag reservoir 25, 225, for example, may be sized to provide sufficient respiratory gas 11 to maintain the respiratory support apparatus in first operational state 92 during inhalation while allowing the respiratory support apparatus to shift from third operational state 96 into fourth operation state 98 during exhalation in order to flush outflow gas 13 from the dead space volume.

The second operational state 94 may be combined with the first operational state 92 to reduce the flow of respiratory gas 11 required to operate the respiratory support apparatus. Recognize from the above discussion that portions of respiratory gas 11 inhaled initially during inhalation are flowed into the alveolar regions of the lungs where O₂ can be absorbed. Other portions of respiratory gas 11 inhaled during later portions of inhalation are flowed into the ADS where O₂ cannot be absorbed. The bag volume of bag reservoir 25 of bag 20, may be selected to sequence communicating respiratory gas 11 with communicating ambient air 12 during inhaling by the user thereby communicating the respiratory gas 11 into alveolar regions of the user as per first operational state 92 and communicating ambient air 12 into the ADS of the user as per second operational state 94. For example, the bag volume may be commensurate with the alveolar volume (e.g., the volume of respiratory gas delivered to the alveolar region).

For example, consider the 70-kg man of Example 1 with a TV of 420 ml and an ADS volume of 140 ml so that the alveolar ventilation comprises about 280 ml. Respiratory support apparatus 10 may be configured so that when the user inhales, respiratory support apparatus 10 initial operates in operational state 92 so that, for example, respiratory gas 11 is delivered into the alveolar regions of the lungs during the first 280 ml inhaled. Respiratory support apparatus 10 then operates in second operational state 94 so that ambient air 12 is communicated through check valve 50c into the anatomical dead space 196 during the remaining 140 ml inhaled. An additional volume of ambient air 12 may be delivered to the dead space volume. This exemplary method of operation, for example, conserves the 140 ml of respiratory gas 11 that would otherwise occupy the anatomical dead space 196 and may conserve an additional 100 ml or so of respiratory gas 11 that would otherwise occupy at least portions of facemask chamber 15 and/or manifold chamber 35, resulting in about 35% to about 50% conservation of respiratory gas 11. Thus, per this example, the available respiratory gas 11 is maximally used for alveolar oxygen exchange in alveolar regions of the lungs and not wasted by being communicated into the ADS and the dead space volume where no oxygen exchange occurs. Communicating ambient air 12 into the ADS of the user may avoid wasting respiratory gas 11, as the user attains no clinical benefit from respiratory gas 11 in the ADS. By contrast, opening of check valve 50c too early during inhalation when there otherwise would be sufficient respiratory gas 11 available flows ambient air 12 into the alveolar region, thereby diluting the respiratory gas being delivered to the patient.

An implementation of the respiratory support apparatus, such as respiratory support apparatus 10, 200, was tested by pulmonologists and intensivists at the University of Texas Health Sciences Center-St. Antonio. Because it is impractically invasive clinically to measure the alveolar oxygen concentration, a simulation using a widely-used Michigan Test Lung driven by a Drager ventilator was used instead. The study compared the instant respiratory support apparatus at 10 LPM oxygen flow against a widely used HFNC, model AIRVO 2 by Fisher & Paykel at 40 and 60 LPM, the commonly used oxygen flow rate for this type of device. Using a tidal volume of 500 ml and RR of 10 (for the minute ventilation of 5 L/min) and 500 ml and RR of 20 (for minute ventilation of 10 L/min), the oxygen concentrations measured in the tracheal region is as shown below:

TABLE 1

| | Minute | Minute |
|---|---|---|
| | Ventilation | Ventilation |
| Device & O2 flow rate | 5 L/min | 10 L/min |
| Respiratory support apparatus | 99.9% | 99.4% |
| implementation at 10 L/min. | | |
| HFNC AIRVO 2 ™ at 40 L/min | 90.1% | 91.4% |
| HFNC AIRVO 2 ™ at 60 L/min | 94.8% | 93.9% |

It can be seen that in spite of saving as much as 83% of the oxygen flow (10 LPM vs 60 LPM), the instant respiratory support apparatus delivers a significantly higher oxygen concentration, but without the costly equipment, personnel and supplies (cannula, heating and humidification coils) needed; nor the discomfort and complications associated with blowing 1000 ml/second up the user's nose. Despite saving 72,000 liters/day of oxygen compared to HFNC at 60 LPM (the standard flow rate), and despite HFNC generally meeting the PIFR demand, the instant respiratory support apparatus has also unexpectedly been shown in test clinical cases to effectively reverse hypoxia to avert ICU admission or intubation in a variety of serious conditions including COVID ARDS and congestive heart failure. Thus, the implementation of the respiratory support apparatus as per Table 1 exhibits surprising and unexpected results.

The combination of second operational state 94 with first operational state 92 for respiratory support apparatus 10, 200 is illustrated in FIG. 9 as exemplary method 500. As illustrated in FIG. 9, exemplary method 500 is entered at step 501. At step 505, a check valve, such as check valve 50a, 250a, is opened as a user is inhaling.

As per step 510, opening the check valve at step 505 allows communication of only a respiratory gas, such as respiratory gas 11, 211, into a manifold chamber, such as manifold chamber 35, 235, of a manifold, such as manifold 30, 230. Only the respiratory gas is then communicated from the manifold chamber into lungs of the user.

At step 515, an anti-asphyxiation valve, such as anti-asphyxiation valve 50c, 250c, is opened as the user is inhaling. The bag volume of the bag reservoir may be sized in relation to an alveolar volume of an alveolar region to open the third check valve to deliver ambient air through the manifold chamber only into the anatomical dead space while avoiding delivery of ambient air into the alveolar regions.

As per step 520, opening of the anti-asphyxiation valve at step 515 allows communication of ambient air, such as ambient air 12, into the manifold chamber and thence into anatomical dead space of the user.

Exemplary method 500 terminates at step 531.

Steps 505, 510 are performed sequentially with steps 515, 520. First, at steps 505, 510, only the respiratory gas is communicated into the manifold chamber and thence into alveolar regions of the lungs of the user. Then, at steps 515, 520, ambient air is communicated from the ambient environment into the manifold chamber and thence into the anatomical dead space of the user. The facemask chamber, such as facemask chamber 15, 215, of the facemask, such as facemask 14, 214, may also be filled, at least in part, with ambient air at the conclusion of steps 515, 520. The manifold chamber may also be filled, at least in part, with ambient air at the conclusion of steps 515, 520.

The combination of third operational state 96 with fourth operational state 98 for respiratory support apparatus 10, 200 is illustrated in FIG. 10 as exemplary method 600. As illustrated in FIG. 10, exemplary method 600 is entered at step 601. At step 605, a check valve, such as check valve 50b, 250b, is opened as a user is exhaling.

As per step 610, opening the check valve at step 605 allows communication of outflow gas, such as outflow gas through the facemask chamber, such as facemask chamber 15, 215, thence through manifold chamber 35, 235, and finally through the open check valve into the ambient environment.

At step 615, another check valve, such as check valve 50a, 250a, is opened as the user is exhaling as prompted by filling of the bag reservoir.

At step 620, respiratory gas passes through the check valve opened at step 615 thence through the manifold chamber and the facemask chamber and finally through the check valve opened at step 605 into the ambient environment thereby flushing outflow gas from the manifold chamber and the facemask chamber into the ambient environment and replenishing the manifold chamber and the facemask chamber with breathable gas.

Exemplary method 600 terminates at step 631.

Steps 605, 610 are performed sequentially with steps 615, 620. First, at steps 605, 610, the respiratory gas is prevented from being communicated into the manifold chamber thereby forcing the respiratory gas to flow into the bag reservoir of the bag to fill the bag reservoir. Filling of the bag reservoir initiates steps 515, 520 in which the respiratory gas then flushes outflow gas from the manifold chamber and the facemask chamber. The manifold chamber and the facemask chamber are then generally filled with respiratory gas at the conclusion of steps 515, 520 so that respiratory gas is then communicated into the alveolar regions upon initiation of the next inhalation, e.g., steps 505, 510 of method 500.

Methods 500, 600 are further elucidated in Example 3 and Example 4 as follows. Note that values in Example 3 and Example 4 are rounded for explanatory purposes

### Example 3

This example follows from Example 1 for a user with an RR of 10 breaths/min or 6 s/breath with inhalation period of 2.4 s and exhalation period 3.6 s. The TV is 420 ml so that the respiratory gas is inhaled on average at 420 ml/2.4 s or 175 ml/s. The respiratory gas flow rate of 5 l/min is equal to 83 ml/s. Accordingly, the bag reservoir provides (175 ml/s-83 ml/s) 2.4 s=220 ml of the respiratory gas being inhaled, which is the volume that the bag reservoir is depleted during inhalation. The bag reservoir refills during exhalation requiring 220 ml/83 ml/s=2.65 s. Upon complete filling of the bag reservoir, the dead space volume is then flushed by respiratory gas (see steps 615, 620 of method 600). The flushing time t_{F} per this Example, which is the time spent in fourth operational state 98, is then (3.6 s-2.65 s)≈1 s. A detention time t_{D} of 1 s would then allow flushing on average of the dead space volume, which results in dead space volume=(83 ml/s)(1 s)≈80 ml. At a flow of 10 l/min and detention time t_{D} of 1 s the dead space volume is then (167 ml/s)(1 s)≈170 ml. Given a manifold volume of 10 ml, the facemask volume (e.g., a functional volume-volume within the facemask with the facemask enclosing the inspiratory aperture of the user) should be generally within the range of 70 ml to 160 ml in order to flush the dead space volume during exhalation for respiratory gas being delivered approximately over a range of from 5 l/min to 10 l/min.

### Example 4

Example 4 follows from Example 2 exemplary of a user in a distressed condition having an RR of 20 breaths/min or 3 s/breath with inhalation period of 1.2 s and exhalation period 1.8 s. The TV is 210 ml so that the respiratory gas is inhaled on average at 210 ml/1.2 s or 175 ml/s. A respiratory gas flow rate of 5 l/min is equal to 83 ml/s. Accordingly, the bag reservoir provides (175 ml/s-83 ml/s) 1.2 s=110 ml of the respiratory gas being inhaled, which is the volume that the bag reservoir is depleted during inhalation. The remainder is provided by flow from the gas source during inhalation. Refilling the bag reservoir during exhalation requires 110 ml/83 ml/s=1.33 s. Upon complete filling of the bag reservoir, the dead space volume is then flushed by respiratory gas for flushing time t_{F}, which is the time during which the respiratory support apparatus is in fourth operational state 98 (see steps 615, 620 of method 600). The dead space volume is then flushed for flushing time t_{F} of (1.8 s-1.3 s)≈0.5 s. A dead space volume of 80 ml then results in a detention time t_{D}=(80 ml)/(83 ml/s)≈1 s so that the dead space volume is flushed for ½ t_{D}. A respiratory gas flow rate of 10 l/min would flush the dead space volume for about 1 t_{D}. A dead space volume of 170 ml would have a t_{D} of about 2 s so that the dead space volume is then flushed for ¼ t_{D} for a flow rate of 5 l/min or ½ t_{D} at a flow rate of 10 l/min.

The change in CO₂ concentration in the dead space volume between an initial concentration C₀ and a final concentration C₁ may be modeled by

$$\frac{{C}_{1}}{{C}_{0}} = {e}^{- {t}_{F} / {t}_{D}} \qquad ( 1 )$$

where t is the time during which the dead space volume is flushed. Flushing for time t_{F} equal to detention time t_{D} reduces the CO₂ concentration in the dead space volume to 0.37 of the CO₂ concentration at the start of flushing. Similarly, flushing for time t_{F} equal to detention time ½ t_{D} reduces the CO₂ concentration in the dead space volume to 0.61 of the CO₂ concentration at the start of flushing, and flushing for time t_{F} equal to detention time ¼ t_{D} reduces the CO₂ concentration in the dead space volume to 0.78 of the CO₂ concentration at the start of flushing.

Thus, given a manifold volume of 10 ml, the facemask volume should be generally within the range of 70 ml to 160 ml in order to flush CO₂ from the dead space volume during exhalation for respiratory gas being delivered approximately over a range of from 5 l/min to 10 l/min. In certain implementations, the dead space volume is less than an alveolar volume of an alveolar region of the user to ensure that least some delivery of respiratory gas untainted by CO₂ to the alveolar volume. Accordingly, the dead space volume may be around 140 ml which is the ADS for an exemplary adult male. In various implementations, the dead space volume may be about 2 ml/kg, for example, in order to take into account uses with children, small adults, or large adults. The ratio of flushing time t_{F} to detention time t_{D}, which is indicative of the amount of flushing, may be determined by the dead space volume, the flow rate of respiratory gas, or the dead space volume and the flow rate of respiratory gas.

The foregoing discussion along with the Figures discloses and describes various exemplary implementations. These implementations are not meant to limit the scope of coverage, but, instead, to assist in understanding the context of the language used in this specification and in the claims. The Abstract is presented to meet requirements of 37 C.F.R. § 1.72 (b) only. Accordingly, the Abstract is not intended to identify key elements of the apparatus and methods disclosed herein or to delineate the scope thereof. Upon study of this disclosure and the exemplary implementations herein, one of ordinary skill in the art may readily recognize that various changes, modifications and variations can be made thereto without departing from the spirit and scope of the inventions as defined in the following claims.

1. A respiratory support apparatus, comprising:
a manifold defining a manifold chamber;
a facemask in mechanical cooperation with the manifold, the facemask defining a facemask chamber in fluid communication with the manifold chamber, the facemask adapted to enclose an inspiratory aperture of a user in order to communicate a respiratory gas into alveolar regions of the user, the respiratory gas comprises oxygen at a concentration greater than that of ambient air, and the user is spontaneously breathing;
a bag defining a bag reservoir in fluid communication with the respiratory gas;
a check valve in cooperation with the manifold, the check valve configured to allow communication of the respiratory gas into the manifold chamber during inhalation, the check valve configured to block communication of the respiratory gas into the manifold chamber to fill the bag reservoir with respiratory gas during a portion of exhalation wherein the bag reservoir is less than completely filled with respiratory gas, the check valve being configured to allow communication of the respiratory gas into the manifold chamber during another portion of exhalation wherein the bag reservoir is completely filled with the respiratory gas;
a second check valve in cooperation with the manifold and configured to allow communication of outflow gas from the manifold chamber to the ambient environment during exhalation and block communication of ambient air from the ambient environment into the manifold chamber during inhalation;
a third check valve in cooperation with the manifold to act as an anti-asphyxiation valve, the third check valve opens to deliver ambient air into the manifold chamber during inhalation by exhaustion of respiratory gas within the bag reservoir;
a first arm defining a first arm passage, a second arm defining a second arm passage, a third arm defining a third arm passage, and a fourth arm defining a fourth arm passage, the first arm, the second arm, and the third arm being coplanar and disposed radially with and perpendicular to the fourth arm, the first arm and the third arm being perpendicular to the second arm, the fourth arm being attachable to a facemask conduit of a facemask to fluidly communicate between the manifold chamber and a facemask chamber of the facemask;
and wherein the manifold chamber of the manifold is bounded by a downstream side of the first check valve received within the first arm passage of the first arm, an upstream side of the second check valve received in the second arm passage of the second arm, and a downstream side of the third check valve received within the third arm passage of the third arm, the downstream side of the first check valve, the upstream side of the second check valve, and the downstream side of the third check valve are positioned proximate a circumference of the fourth arm passage in order to minimize a manifold volume of the manifold chamber.

2. The apparatus of claim 1, wherein a manifold volume of the manifold chamber is about 10 ml.

3. The apparatus of claim 2, wherein a manifold detention time of the manifold is less than about 0.12 s.

4. The apparatus of claim 1, further comprising:
a dead space volume comprising the manifold volume of the manifold chamber and a facemask volume of the facemask chamber with the facemask enclosing the inspiratory aperture of the user, wherein the dead space volume is less than an alveolar volume of an alveolar region of the user.

5. The apparatus of claim 1, further comprising:
a dead space volume comprising a manifold volume of the manifold chamber and a facemask volume of the facemask chamber with the facemask enclosing the inspiratory aperture of the user, wherein the dead space volume is within a range of from about 70 ml to about 160 ml.

6. The respiratory support apparatus of claim 1, further comprising:
a dead space volume comprising a manifold volume of the manifold chamber and a facemask volume of the facemask chamber with the facemask enclosing the inspiratory aperture of the user, wherein the dead space volume is about 100 ml.

7. The respiratory support apparatus of claim 1, further comprising:
a detention time of said respiratory support apparatus wherein the detention time is less than about 1.2 s, the detention time being defined as the dead space volume divided by a flow rate of the respiratory gas flowing through the dead space volume from the first check valve through the second check valve, the dead space volume comprising a manifold volume of the manifold chamber and a facemask volume of the facemask chamber with the facemask enclosing the inspiratory aperture of the user.

8. The respiratory support apparatus of claim 1, further comprising:
a detention time of said respiratory support apparatus wherein the detention time is less than about 2.4 s, the detention time being defined as the dead space volume divided by a flow rate of the respiratory gas flowing through the dead space volume from the first check valve through the second check valve, the dead space volume comprising a manifold volume of the manifold chamber and a facemask volume of the facemask chamber with the facemask enclosing the inspiratory aperture of the user.

9. The respiratory support apparatus of claim 1,
wherein a facemask volume of the facemask chamber with the facemask enclosing the inspiratory aperture of the user is less than about 100 ml.

10. The respiratory support apparatus of claim 1, further comprising a mask with a sidewall having a concave shape configured to approximate a facial contour of a face.

11. The respiratory support apparatus of claim 1, wherein a bag volume of the bag reservoir is sized in relation to an alveolar volume of an alveolar region to open the third check valve to deliver ambient air through the manifold chamber only into the anatomical dead space while avoiding delivery of ambient air into the alveolar regions.

13. The respiratory support apparatus of claim 1, further comprising:
a sensor in communication with the manifold chamber through a sensor passage, the sensor configured to monitor an attribute of the outflow gas only during exhalation and only while outflow gas is flowing out of the manifold chamber and no respiratory gas is flowing into the manifold chamber.

14. The respiratory support apparatus of claim 1, wherein a bag volume of the bag reservoir is sized to be greater than twice a tidal volume of the user.

15. The respiratory support apparatus of claim 1, wherein a flow rate of the respiratory gas flowing from a gas source and a dead space volume comprising the manifold volume of the manifold chamber and a facemask volume of the facemask chamber with the facemask enclosing the inspiratory aperture of the user are configured to equate generally a detention time and a flushing time.

16. A method of respiratory support, comprising the steps of:
allowing communicating of a respiratory gas into a manifold chamber of a manifold during inhalation by a check valve that cooperates with the manifold, the manifold attached to a facemask that defines a facemask chamber, the facemask adapted to enclose an inspiratory aperture of a user in order to communicate a respiratory gas at least into alveolar regions of the user, the respiratory gas comprises oxygen at a concentration greater than that of ambient air;
blocking communicating of the respiratory gas into the manifold chamber by the check valve thereby filling a bag reservoir of a bag with the respiratory gas during a portion of exhalation, the bag reservoir being less than completely filled with the respiratory gas;
allowing communicating of the respiratory gas into the manifold chamber by the check valve during another portion of exhalation thereby flushing an outflow gas from the manifold chamber, the bag reservoir being completely filled with the respiratory gas;
allowing communicating of the outflow gas from the manifold chamber into an ambient environment during exhalation by a second check valve that cooperates with the manifold;
blocking communicating of ambient air from the ambient environment into the manifold chamber during by the second check valve during inhalation;
and controlling communicating of ambient air into the manifold chamber during inhalation using a third check valve that cooperates with the manifold, the third check valve acting as an anti-asphyxiation valve, the third check valve in combination with a bag volume of the bag reservoir being configured to deliver ambient air through the manifold chamber only into the anatomical dead space while avoiding delivery of ambient air into the alveolar regions.
